SEQUENCE LISTING (12 sequences)

SEQ ID NO: 1 - DNA, 4395 bp, Homo sapiens
  CDS (564)..(4166): Homo sapiens
ggcgaggatggggagcaatgcaaagctctgctcgccccccatctcaccccccaagcggatactggtcttctcgtcggattgcccatgcacttgttgcagaaacagccaaggccctggctgtggagaatgctgaaggaagaagacgcagaagcaggacgaccctgaaagattcagcctcttcatcctcaaacaggaccaggcctcctcttcccacctgtccagcagcatgaaagcagcatgattggccgaccgcaggagaagcccccagaaccaggcccccaactcagccatctgcggaggtcaaggtgtgagcgacgtctcctcaccacagtgctgtgtggtctatacctcagccagggagaggatgtgaaaccccccgccctgcacatgagtggtacaggccaacaggaacacctggctccagccacgttcacagacatgtcagccgtggagtagtgctgacacttttctctcagcttctcagggtttcagtccttttgggtttgttttatttaccttttttatggttttgtggctggacgttcacaaccaaggcagacagcatgggtgaccagcaactgtacaagaccaaccatgtggcccatggtagtgagaaccttttctaccaacagccaccacttggcgtccacagcgggctgaaccacaactatgggaatgcagttacagggggcggaatggatgcccctcaggcctcgccaatctccccccacttccctcaagatacacgggatggtctgggcttgcctgttggctccaaaaaccttggccaaatggatacctcgaggcagggagggtggggaagtcatgcagggcctggaaaccatgtccagctacgtggaaacctggccaactcaaacatgatgtggggggcaccagcccaggctgagcccactgatggctaccaatacacctactcccaggccagcgagatccggacccagaagcttaccagcggtgtcttacacaagctggactctttcacccaggtgtttgccaaccaaaacctgcgaattcaggtcaacaatatggcccaggtgctgcacactcagtcagcagtgatggatggagcccctgacagtgctctccgccagctgctgtctcagaagcccatggagcccccagcaccggctatcccttcccgctaccagcaggtgccccagcagcctcaccctggtttcactggtgggctgtccaaaccagctcttcaggtcgggcagcaccctacccaagggcacctgtattatgactaccagcagcctctggctcaggtgccagtgcagggaggacagccactgcaggccccacagatgctgtcacagcacatgcaacagatgcagcagcaccagtattacccaccgcagcaacagcagcaagccgggcaacagcgtatctccatgcaagaaatacagacgcagccgcaacaaattcgcccatcacagccacagccgccgccacagcagcagcagccgcagcagctacagctgcagcagcggcagggttcaatgcagatacctcagtattatcagccccaacccatgatgcagcacttgcaagagcagcagcagcaacagatgcacctgcagcctccttcttatcacagggaccctcaccagtataccccagagcaggcacacactgtccagctgattcccctgggctccatgtcccagtactactaccaggagccccagcagccctacagccaccccctctaccagcagagccacctgtcccagcaccagcagcgtgaggacagtcagctgaagacctactctagtgacagacaggcccaggccatgctgagctcccatggggacctggggcctcctgacacaggaatgggagacccagcgagctcagatctgacccgggtcagcagcaccctcccccatcgccccctcctatcccccagtgggatccacctcaacaacatggggcctcagcatcagcagctgtctcccagtgccatgtggccccagatgcacctacctgatgggagagcccagccagggtcccctgagtcaagtggccaacccaaaggagcgtttggggagcagtttgatgccaagaacaagctgacatgctccatctgcctgaaggagttcaagaacctgcctgccctgaatggccacatgcggtcccacgggggaatgagggcctcccccaacctcaaacaggaggaaggagagaaggtcctgccgcctcagccccagccaccactgccgcctccgcctccgcctccgccgccaccacagctccctcccgaggcagaaagcctcacgcctatggtcatgcccgtgtctgtccctgtcaagcttctcccgcccaagcccagctctcaggggttcaccaacagcaccgttgccgccccctccgccagagacaagccagccagctcgatgtcggacgacgagatgcctgtgctcgaaatccccaggaagcatcagccgagtgtgcccaaagccgaggagcccctcaagaccgtgcaggagaagaaaaagttccggcaccggccggaacctctcttcatcccgccgccgccctcctacaacccgaaccccgctgcctcctactcgggcgccaccctgtaccagagccagctgcgctccccgcgcgtcctcggggaccacctgctcctggaccccacccacgagctgcccccttacacgcccccacccatgctgagcccggtgcgccagggctcggggctcttcagcaatgtcctcatctccggccacggccctggcgcccacccgcagctgcccctgacgcccctgacgcccacaccacgggtgctgctgtgtcgctccaacagcatcgatggcagcaacgtgacggtcaccccagggcctggagagcagactgtagatgttgaaccacgcatcaacattggcttgagattccaagcagaaatccctgaactccaagatatctctgccctggcccaggacacacacaaggccacactggtatggaagccctggccagaactagaaaaccatgacctccagcaaagagtggagaatcttctgaatttgtgctgttccagtgcattgccaggtggagggaccaattctgaatttgctttgcactctctgtttgaggccaaaggtgatgtgatggttgctctggaaatgctgctactgcggaagcctgtcaggttaaaatgtcatcctttagcaaattaccactatgccggttcggacaagtggacctccctagaaagaaaactgtttaacaaagcactagccacttacagcaaagactttatttttgtacagaagatggtgaagtccaagacggtggctcagtgcgtggagtactactacacgtggaaaaagatcatgcggctggggcggaaacaccggacacgcctggcagaaatcatcgacgattgtgtgacaagtgaagaagaagaagagttagaggaggaggaggaggaggacccggaagaagataggaaatccacaaaagaagaagagagtgaggtgccgaagtccccggagccaccacccgtccccgtcctggctcccacggaggggccgcccctgcaggccctgggccagccctcaggctccttcatctgtgaaatgcccaactgtggggctgtgttcagctcccgacaggcactgaatggccatgcccgcatccacgggggcaccaaccaggtgaccaaggcccgaggtgccatcccctctgggaagcagaagcctggtggcacccagagtgggtactgttcggtaaagagctcaccctctcacagcaccaccagcggcgagacagaccccaccaccatcttcccctgcaaggagtgtggcaaagtcttcttcaagatcaaaagccgaaatgcacacatgaaaactcacaggcagcaggaggaacaacagaggcaaaaggctcagaaggcggcttttgcagctgagatggcagccacgattgagaggactacggggcccgtgggggcgccggggctgctgcccctggaccagctgagtctgatcaaacccatcaaggatgtggacatcctcgacgacgacgtcgtccagcagttgggaggtgtcatggaagaggctgaagttgtggacaccgatcttctcttggatgatcaagattcagtcttgcttcagggtgacgcagaactataaagccctgtgtgtcacttagagacagtgaaaacccacggcctccatcttcattaatcaggaaacctggactgcctgcttgttttgtaacccttttaaactacctgttttaaaagtggtcattttattcaggtttagaaaaaaaaatcctatttcttttcttttatttaaaaaaatttgtttttgtggggggttgggggaataaataattggcacaactaaaaaaaaaaaa

SEQ ID NO: 2 - protein, 1200 aa, Homo sapiens
MGDQQLYKTNHVAHGSENLFYQQPPLGVHSGLNHNYGNAVTGGGMDAPQASPISPHFPQDTRDGLGLPVGSKNLGQMDTSRQGGWGSHAGPGNHVQLRGNLANSNMMWGAPAQAEPTDGYQYTYSQASEIRTQKLTSGVLHKLDSFTQVFANQNLRIQVNNMAQVLHTQSAVMDGAPDSALRQLLSQKPMEPPAPAIPSRYQQVPQQPHPGFTGGLSKPALQVGQHPTQGHLYYDYQQPLAQVPVQGGQPLQAPQMLSQHMQQMQQHQYYPPQQQQQAGQQRISMQEIQTQPQQIRPSQPQPPPQQQQPQQLQLQQRQGSMQIPQYYQPQPMMQHLQEQQQQQMHLQPPSYHRDPHQYTPEQAHTVQLIPLGSMSQYYYQEPQQPYSHPLYQQSHLSQHQQREDSQLKTYSSDRQAQAMLSSHGDLGPPDTGMGDPASSDLTRVSSTLPHRPLLSPSGIHLNNMGPQHQQLSPSAMWPQMHLPDGRAQPGSPESSGQPKGAFGEQFDAKNKLTCSICLKEFKNLPALNGHMRSHGGMRASPNLKQEEGEKVLPPQPQPPLPPPPPPPPPPQLPPEAESLTPMVMPVSVPVKLLPPKPSSQGFTNSTVAAPSARDKPASSMSDDEMPVLEIPRKHQPSVPKAEEPLKTVQEKKKFRHRPEPLFIPPPPSYNPNPAASYSGATLYQSQLRSPRVLGDHLLLDPTHELPPYTPPPMLSPVRQGSGLFSNVLISGHGPGAHPQLPLTPLTPTPRVLLCRSNSIDGSNVTVTPGPGEQTVDVEPRINIGLRFQAEIPELQDISALAQDTHKATLVWKPWPELENHDLQQRVENLLNLCCSSALPGGGTNSEFALHSLFEAKGDVMVALEMLLLRKPVRLKCHPLANYHYAGSDKWTSLERKLFNKALATYSKDFIFVQKMVKSKTVAQCVEYYYTWKKIMRLGRKHRTRLAEIIDDCVTSEEEEELEEEEEEDPEEDRKSTKEEESEVPKSPEPPPVPVLAPTEGPPLQALGQPSGSFICEMPNCGAVFSSRQALNGHARIHGGTNQVTKARGAIPSGKQKPGGTQSGYCSVKSSPSHSTTSGETDPTTIFPCKECGKVFFKIKSRNAHMKTHRQQEEQQRQKAQKAAFAAEMAATIERTTGPVGAPGLLPLDQLSLIKPIKDVDILDDDVVQQLGGVMEEAEVVDTDLLLDDQDSVLLQGDAEL

SEQ ID NO: 3 - DNA, 21 bp, artificial sequence
  primer
ggcactcttgttcccccaggc

SEQ ID NO: 4 - DNA, 20 bp, artificial sequence
  primer
accatccccttcctcacctg

SEQ ID NO: 5 - DNA, 21 bp, artificial sequence
  primer
gcacactgacgcagcacacag

SEQ ID NO: 6 - DNA, 21 bp, artificial sequence
  primer
cagtttgagaagcagccacct

SEQ ID NO: 7 - DNA, 22 bp, artificial sequence
  primer
aggccagccagagcaggtttgt

SEQ ID NO: 8 - DNA, 22 bp, artificial sequence
  primer
ggaggagatccattggttgcgg

SEQ ID NO: 9 - DNA, 25 bp, artificial sequence
  primer
gacttgcatctagtcctgactccgg

SEQ ID NO: 10 - DNA, 25 bp, artificial sequence
  primer
gcctacctcatctcatacgctccag

SEQ ID NO: 11 - DNA, 23 bp, artificial sequence
  primer
aaactctccctcctcctcttcct

SEQ ID NO: 12 - DNA, 23 bp, artificial sequence
  primer
cgagccataaaaggcaactttcg

(19) United States
(12) Patent Application Publication
(10) Pub. No.: US 2008/0044436 A1
(43) Pub. Date: Feb. 21, 2008

(54) Method for Screening and Compositions for Treating Cancers

(76) Inventors: Dean W. Hum, Bondues (FR); Florence Gizard, Lille (FR)

Correspondence Address: NIXON & VANDERHYE, PC, 901 NORTH GLEBE ROAD, 11TH FLOOR, ARLINGTON, VA, 22203, US

(21) Appl. No.: 11/597,633

(22) PCT Filed: May 27, 2005
(86) PCT No.: PCT/FR05/01314

§ 371 Date: Nov. 27, 2006

(30) Foreign Application Priority Data
May 27, 2004 (FR) 0405737

(51) Int. Cl. A61K 38/00; A61P 37/00; C12Q 1/68; G01N 33/53

(52) U.S. Cl. 424/198.1; 435/6; 435/7.1

(57) ABSTRACT

The invention relates to compositions and methods for treating proliferative pathologies, in particular cancers. The invention is based on the identification of particular properties of TReP-132 in the control of the cell cycle and in the control of cell proliferation. Said protein behaves as a tumor suppressor and is therefore a preferred target for developing efficient therapeutic strategies. The invention also relates to compositions, uses and methods for identifying compounds which can modulate the activity of said protein, and which can be used for treating different proliferative pathologies, in particular in humans.

The invention relates to compositions and methods for treating proliferative pathologies, in particular cancers and more particularly hormone-dependent cancers. The invention is based on the identification of particular properties of a protein in the control of the cell cycle and in the control of cell proliferation. Said protein, named TReP-132, behaves as a tumor suppressor and is therefore a preferred target for developing efficient therapeutic strategies. The invention also relates to compositions, uses and methods for identifying compounds which can modulate the activity of said protein and which can be used for treating different proliferative pathologies, in particular in humans.

The functional characterization of the cytochrome P450scc gene promoter led to the isolation of the TReP-132 protein (Transcriptional Regulating Protein of 132 kDa). Said protein is involved in regulating the expression of steroidogenic genes and in particular in activating the expression of the gene coding for the P450scc enzyme (Gizard et al., 2001 and Gizard et al., 2004). Cytochrome P450scc is a mitochondrial enzyme encoded by the CYP11 A1 gene and expressed in steroidogenic tissues such as ovary, testis, placenta or adrenal glands (Mellon et al., 1993 and Stromstedt et al., 1995). P450scc catalyzes the first enzymatic step in steroid synthesis by converting cholesterol to pregnenolone (Miller, 1988).

Overexpression of TReP-132 in human adrenal NCI-H295 cells has been shown to result in a significant increase in pregnenolone production, which is in agreement with the ability of said factor to increase P450scc gene expression and potentially increase the production of steroid hormones, including mineral corticoids, glucocorticoids and sex steroids.

TReP-132 therefore appears to be involved in multiple physiological effects of steroid hormones.

TReP-132 cDNA (3600 nucleotides) was first isolated by screening a human placenta cDNA library. The protein sequence displays the characteristic motifs of a transcriptional regulatory factor, such as three zinc finger motifs of the C₂H₂ subtype, regions rich in acidic amino acids, proline and glutamine, and two LXXLL motifs. Northern blot and RNase protection assays showed that TReP-132 is expressed in different tissues, with particularly high expression in steroid target tissues such as uterus, prostate, testis and certain areas of the brain (Gizard et al., 2001; Gizard et al., 2002b and Duguay et al., 2003) as well as in the cancer cell lines LNCaP, MCF-7 and T47-D (Gizard et al., 2001). Immunocytochemical studies localized the TReP-132 protein to the nucleus. Based on the primary structure analysis, it appears that TReP-132 contains several protein interaction domains such as two LXXLL motifs. Studies in NCI-H295 cells showed that the regulation of gene expression by TReP-132 involves the formation of a transcription activation complex and a direct interaction of TReP-132 with SF-1 (Steroidogenic Factor-1) and the cofactor CBP/p300 (Gizard et al., 2002b and Gizard et al., 2004). The TReP-132/SF-1 interaction requires the LXXLL motif located in the N-terminal region of TReP-132 as well as the proximal activation domain and the AF-2 motif of the SF-1 protein.

The interaction of TReP-132 with DNA, the nuclear localization thereof and the involvement thereof in the regulation of gene expression indicate that TReP-132 is a transcriptional regulatory factor.

The inventors have found that TReP-32 is expressed in different tissues, including non-steroidogenic tissues such as brain, thymus and heart, which, considering its ability to interact with DNA to regulate gene expression, seems to indicate that TReP-132 may be involved in different functions, other than the regulation of steroid synthesis. Accordingly, the application now demonstrates the involvement of TReP-132 in cell proliferation and associated mechanisms, such as cell differentiation and tissue development. In particular, the application demonstrates a regulation of TReP-132 expression during the cell cycle, and reveals a role of said factor in regulating cell proliferation.

More particularly, the inventors have shown that TReP-132 is involved in cell proliferation dependent on steroid hormones or analogs thereof and more specifically progesterone, particularly in cancers affecting steroid hormone-responsive organs and typically female cancers, in particular breast and uterine cancer. In fact, steroid hormones (estrogen and progesterone) play a key role in mammary gland proliferation and differentiation (Musgrove et al., 1994). In vitro studies in T47-D cell lines have shown that the cellular response to progesterone or its derivatives R5020 and ORG 2058 (Stahlberg et al., 2003) is biphasic: initial exposure leads to proliferation whereas prolonged exposure results in inhibition of cell growth. Thus, long-term therapy with synthetic progestins such as megestrol acetate or medroxy-progesterone acetate is used clinically in the treatment of certain breast cancers (Sitruk-Ware et al., 1999 and Ingle, 2002).

The application demonstrates the role of TReP-132 as a regulator of cell proliferation. The inventors have shown that transfection of HeLa cells, which do not endogenously express PR (Progesterone Receptor), with a TReP-132 expression vector leads to fewer colonies than transfection with an empty vector alone. This finding indicates that TReP-132 expression decreases or arrests cell cycle progression. Furthermore, cells expressing exogenous TReP-132 have a significantly longer doubling time than control cells and the colonies obtained after TReP-132 transfection are smaller.

In parallel, the inventors have shown that TReP-132 expression is increased in T47-D cells after treatment with progesterone and that selective inhibition of TReP-132 increases cell proliferation and abolishes the inhibitory effect of progesterone. These findings demonstrate the inhibitory effect of TReP-132 on progesterone-dependent cell proliferation. The involvement of TReP-132 in cell proliferation was subsequently demonstrated in human breast cancer MCF-7 epithelial cells: treatment of said cells with estradiol led to a significant decrease in TReP-132 expression and a concomitant increase in cell proliferation. These findings demonstrate the inhibitory effect of TReP-132 on progesterone-dependent and progesterone-independent cell proliferation.

In addition, the application demonstrates a regulation of TReP-132 expression during the cell cycle. It was shown that in the absence of estradiol treatment, a higher proportion of MCF-7 cells were in G₁ phase, thereby suggesting that TReP-132 expression arrests cell cycle progression from G₁ to S phase.

The involvement of TReP-132 in the G₁ phase of the cell cycle was further demonstrated by the finding that said protein was expressed exclusively during the G₁ phase of the cell cycle following synchronization of HeLa cells.

In addition, the inventors have shown that overexpression of TReP-132 in T47-D cells induced an arrest of cell proliferation in the G₁ phase together with an inhibition of CDK (Cyclin Dependent Kinase) activity and a decrease in pRb phosphorylation (Retinoblastoma protein). These findings reveal a negative correlation between TReP-132 expression and cell cycle progression.

The effects of TReP-132 overexpression on the level of cyclin-dependent kinase inhibitors (CDKIs) have also been determined. In so far as TReP-132 has been shown to be involved in regulating gene expression, it was of interest to identify target genes regulated by TReP-132 which are involved in the cell cycle. RNase protection and reporter gene assays showed that induction of TReP-132 expression is associated with an increased expression of the CDKIs p21, p16 and p27. The inventors have also performed transfection, immunoprecipitation and gel shift studies showing that TReP-132 activates the p21, p16 and p27 gene promoters through a mechanism involving a complex formed by TReP-132, the Sp1 transcription factor and PR. These data therefore indicate that TReP-132 acts as a cofactor of PR (Progesterone Receptor), in the regulatory complex formed with Sp1.

Likewise, the inventors have found an interaction between TReP-132 and the estrogen receptor (ER).

The inventors further suggest that TReP-132 expression is associated with a low tumor incidence and low tumor aggressiveness. Interestingly, it has been shown that chromosome 6, which carries the TReP-132 gene in position 6p21.1-p12.1 (Gizard et al., 2002a), is the fourth most frequently rearranged chromosome in human tumors and that the 6p21 locus is a hot spot for mutations associated with immortalizing events in some cancers. Mutations altering the expression or activity of TReP-132 might therefore be associated with tumor development. TReP-132 might therefore be a marker of predisposition, cell differentiation and development, and tumor aggressiveness.

The application also demonstrates the role of TReP-132 in cell differentiation. Gizard et al. (2004) suggest that TReP-132 overexpression in human adrenal NCI-H295 cells induces the differentiation of zonae fasciculata and reticularis cells and that TReP-132 induces the expression of the P450 aromatase gene required for mammary gland differentiation. Moreover, inhibition of T47-D cell proliferation by progesterone, which the inventors have shown increases TReP-132 expression, is related to the induction of differentiation programs (Musgrove et al., 1998). This hypothesis was confirmed by the up-regulation of p21, p16 and p27 which are molecules that facilitate hormone-dependent differentiation in many cell differentiation pathways (Inoue et al., 1999). In breast tissue, p27 up-regulation underlies the arrest of cell differentiation at the alveolar phase (Said et al., 2001) and high levels of p21 and p27 are respectively associated with intermediate and advanced states of differentiation in ductal carcinoma in situ (DCIS—a non-invasive form of breast cancer) (Mommers et al., 2001). Considering that TReP-132 and progesterone have similar effects on the expression of p21, p27 and p16, it clearly appears that TReP-132 is a marker of cell differentiation, in particular of breast epithelial cells, and that TReP-132 is a differentiation factor.

The activities of TReP-132 so demonstrated indicate that the TReP-132 gene is potentially a tumor suppressor gene. As shown for other tumor suppressor genes such as p53 which also activates cyclin-dependent kinase inhibitor genes like p21, TReP-132 probably also regulates the cell cycle through many growth inhibition pathways. This includes positive transcriptional regulation of target genes by promoting apoptosis and of growth inhibitor genes, as well as negative regulation of other genes, including survival factors and anti-apoptosis genes.

Taken together, these data show that the TReP-132 protein can regulate cell cycle progression in different cell types. TReP-132 is therefore a potential tumor suppressor gene. The regulated expression of TReP-132 and the production of functional TReP-132 protein are critical steps required for proper cell cycle progression and constitute particularly advantageous targets for therapeutic intervention. These data also show that compounds which can increase the activity of the TReP-132 protein potentially represent potent and selective inhibitors of the pathways involved in cell cycle progression, uncontrolled cell proliferation, tissue development and/or cell differentiation.

A first object of the invention is based on the use of a compound increasing or mimicking the activity of the TReP-132 protein for preparing a composition intended for the treatment of a proliferative disease.

Another object of the invention relates to the use of said composition in combination with a hormone for a simultaneous, separate or sequential administration.

The invention also relates to methods for treating proliferative pathologies, comprising administering to a subject a compound increasing or mimicking the activity of the TReP-132 protein, in particular a compound which mimics the activity of the TReP-132 protein, stimulates the expression, maturation or nuclear targeting of the TReP-132 protein and/or increases the concentration of the TReP-132 protein in cells. The administration can be carried out by any classical route for this type of therapeutic approach, such as in particular the systemic route, in particular, by injection, particularly intravenous, intradermal, intratumoral, subcutaneous, intraperitoneal, intramuscular, intra-arterial, etc.

A particular object of the invention is based on the use of the TReP-132 protein or an analog for preparing a composition intended for the treatment of a proliferative disease.

Another object of the invention relates to the use of a nucleic acid coding for the TReP-132 protein for preparing a composition intended for the treatment of a proliferative disease.

Another particular object of the invention relates to a pharmaceutical composition comprising a TReP-132 protein or a nucleic acid coding for the TReP-132 protein, and a pharmaceutically acceptable excipient. The invention also relates to a pharmaceutical composition comprising, in a pharmaceutically acceptable excipient, a compound selected in the group consisting of the TReP-132 protein, an analog of same, a compound increasing or mimicking the activity of TReP-132 and a compound identified with the aid of a method according to the invention, combined with one or more hormones, in view of a simultaneous, separate or sequential administration in order to treat a proliferative disease.

In a preferred embodiment, the pharmaceutical composition comprises, in a pharmaceutically acceptable excipient, the TReP-132 protein.

In another preferred embodiment, the pharmaceutical composition comprises, in a pharmaceutically acceptable excipient, progesterone or an estrogen.

The invention can be used for the treatment of various proliferative pathologies, and in particular for the treatment of cancers. The invention is particularly adapted to the treatment of hormone-dependent cancers, in particular cancers which are sensitive to a deregulation in the synthesis and/or activity of estrogens or progesterone. In particular, the invention can be used for the treatment of breast or uterine cancer.

Another object of the application relates to a method for the selection, identification, characterization or optimization of active compounds modulating, preferably reducing, cell proliferation comprising determining the ability of a test compound to mimic, increase or promote the binding of the TReP-132 protein or of a complex comprising the TReP-132 protein to the receptor of one or more steroid hormones or to modulate the activity of a target gene of said receptor, in particular the p27, p21 or p16 gene.

The invention further relates to a method for the selection, identification, characterization or optimization of active compounds, comprising determining the ability of a test compound to modulate the binding of the TReP-132 protein or of a complex comprising the TReP-132 protein to the receptor of one or more steroid hormones. Said method for the selection, identification, characterization or optimization, in vitro or ex vivo, of active compounds modulating cell proliferation, comprises:
a) contacting a test compound with:
   i) the TReP-132 protein or a complex comprising the TReP-132 protein, and
   ii) the receptor of one or more steroid hormones, and
b) measuring the binding of the TReP-132 protein or of the complex comprising the TReP-132 protein to said receptor.

In the context of the invention, the receptor of one or more steroid hormones is in particular the progesterone receptor (PR) or the estrogen receptor (ER).

In particular, the invention relates to a test compound which increases or promotes the binding of the TReP-132 protein or of a complex comprising the TReP-132 protein to the receptor of one or more steroid hormones (for example the progesterone receptor), or else a test compound which reduces or inhibits the binding of the TReP-132 protein or of a complex comprising the TReP-132 protein to the receptor of one or more steroid hormones (for example the estrogen receptor).

Another object of the invention is based on a pharmaceutical composition comprising, in a pharmaceutically acceptable excipient, a compound modulating the binding of the TReP-132 protein to the receptor of one or more steroid hormones.

The application also relates to a method for the selection, identification, characterization or optimization, in vitro or ex vivo, of active compounds modulating, preferably reducing, cell proliferation, comprising:

a) contacting, in the presence of progesterone or estrogens, a test compound with:

i) respectively, the progesterone or estrogen receptor,
ii) Sp1, and
iii) a nucleic acid comprising all or part of the promoter of a target gene of progesterone or estrogens, and
   b) measuring the binding of Sp1 or of the complex comprising the progesterone or estrogen receptor and Sp1 to said promoter, or measuring the expression of said promoter, said measurement being an indication of the effect of the test compound on cell proliferation.

According to another embodiment of the invention, the method is carried out in the presence of the TReP-132 protein or a complex comprising the TReP-132 protein.

In a preferred manner, said method can be carried out in a cell.

In the context of the invention, the target genes of progesterone or estrogens are the p21, p16 and/or p27 genes.

Another aspect of the invention relates to a method for detecting a predisposition, diagnosing, monitoring and measuring the aggressiveness of a proliferative disease comprising detecting, in a patient, the overexpression of estradiol, the reduction in progesterone expression or a decrease in the expression of the CDKIs p21, p16 and/or p27.

As indicated, the invention is based on the demonstration that the TReP-132 protein can act as a cell cycle regulator, and in particular can inhibit the proliferation of cancer cells, in particular in hormone-dependent cancers.

In the context of the invention, the pharmaceutical compositions comprise in particular compounds increasing the binding of the TReP-132 protein to the receptor of one or more steroid hormones, in a pharmaceutically acceptable excipient.

The invention also relates to the use of a compound increasing, promoting or facilitating the binding of the TReP-132 protein to the receptor of one or more steroid hormones for preparing a composition intended for the treatment a proliferative disease.

Another particular object of the invention relates to a method for preparing a pharmaceutical composition for treating a proliferative disease comprising a step which employs a method such as described herienabove.

A particular object of the invention relates to the treatment of different proliferative pathologies, such as cancer or stenosis, advantageously cancer. In particular, the invention can be used in the treatment of hormone-dependent cancers, and in particular in the treatment of breast cancer or uterine cancer.

In the context of the invention, the receptor of one or more steroid hormones is in particular a progesterone receptor or an estrogen receptor.

TReP-132 Protein

In the spirit of the invention, the expression “TReP-132 protein” denotes a polypeptide comprising the sequence SEQ ID NO: 2, the natural or functional variants, derivatives or homologs thereof. More particularly, it is any natural variant of sequence SEQ ID NO: 2, resulting from a polymorphism, splicing, mutation, etc. Said natural variants can therefore comprise one or more modifications such as substitutions, insertions and/or deletions of one or more residues, etc. The term homolog should be understood to mean TReP-132 proteins from other species, for example rodents, bovines, etc. Preferably, it is a polypeptide recognized by a polyclonal antibody produced from the TReP-132 protein corresponding to sequence SEQ ID NO: 2.

In general, the term TReP-132 protein denotes a polypeptide having sequence SEQ ID NO: 2.

Preferred variants contain at least 80% of sequence SEQ ID NO: 2, more preferably at least 90% identity with sequence SEQ ID NO: 2. The degree of identify can be determined by example by using the CLUSTAL method. Particular variants contain a mutation or substitution affecting at most five amino acids of sequence SEQ ID NO: 2. In the spirit of the invention, particular functional variants are variants resulting from splicing event(s), for example the variants described in GenBank under the reference numbers AJ277276 and AJ277275.

Particular functional variants according to the invention are fragments of the TReP-132 protein described hereinabove, in particular polypeptides comprising part of the sequence SEQ ID NO: 2. The polypeptide fragments of the invention preferably contain fewer than 200 amino acids, more preferably fewer than 150 amino acids. A TReP-132 protein according to the invention can also comprise heterologous residues, added to the indicated amino acid sequence. Thus, one object of the invention is a polypeptide comprising all or part of sequence SEQ ID NO: 2 or a natural variant thereof and a heterologous part. The heterologous part can correspond to amino acids, lipids, sugars, etc. It can also be chemical, enzymatic, radioactive group(s), etc. In particular, the heterologous part can be a marker, targeting agent, stabilizer, agent facilitating production, a protective agent, an agent facilitating entry of the protein into cells, a toxin, an active compound, an antibody, etc.

In a general manner, the expression “TReP-132 protein” designates a polypeptide corresponding to SEQ ID NO: 2 or any polypeptide coded by a nucleic acid which hybridizes with sequence SEQ ID NO: 1 or with a region thereof (typically comprising at least 50 bases), in conditions of moderate or high stringency. Suitable stringency conditions are for example an incubation at 42° C. for 12 hours in a solution comprising 50% formamide, 5×SSPE, 5×Denhardt's, 0.1% SDS, (1×SSPE is composed of 0.15 M NaCl, 10 mM NaH₂PO₄, 1.3 mM EDTA, pH 7.4). Of course it is possible to vary the temperature and salt concentration of the medium.

In the context of the invention, the term TReP-132 protein analog denotes any functional analog of the TReP-132 protein. Said functional analogs are polypeptides, optionally comprising a heterologous part such as defined hereinabove, displaying at least one biological property of the TReP-132 protein, in particular the ability to bind a protein, preferably a hormone receptor and in particular a progesterone receptor or an estrogen receptor, or else the ability to regulate the expression of the p27, p21 and p16 genes. In particular, a TReP-132 protein or variant according to the invention comprises at least one LXXLL motif.

In the spirit of the invention, the term “complex comprising the TReP-132 protein” denotes any type of complex in which the TReP-132 protein is involved. Said complex can comprise many partners, such as nuclear receptors, cofactors, and the like, which can have different natures and functions. Cofactors can be exemplified by transcription factors, in particular p300, Sp1 or VDR (Vitamin D Receptor). Receptors are in particular nuclear receptors among which one can mention the receptors of one or more steroid hormones, orphan receptors, the PPAR, LXR, RXR, FXR receptors or any other nuclear receptor. In a preferred manner, the receptors according to the invention are involved in cell proliferation or differentiation. More preferably, the receptors of one or more steroid hormones are the progesterone receptor (PR) and the estrogen receptor (ER).

A TReP-132 protein according to the invention can be prepared by any method known to those skilled in the art, in particular by artificial synthesis and more particularly by solid phase synthesis, or by any biological, genetic or enzymatic method, and in particular by expression of a nucleic acid encoding said protein in a suitable host cell. In the context of the invention, a preferred TReP-132 protein is a human TreP-132 protein having a sequence identical to sequence SEQ ID NO: 2 for example.

Compound

An object of the invention is based on a method for the selection, identification, characterization or optimization of active compounds modulating, preferably reducing, cell proliferation, comprising determining the ability of a test compound to mimic, increase or promote the binding of the TReP-132 protein or of a complex comprising the TReP-132 protein to the receptor of one or more steroid hormones.

Said method can comprise contacting a test compound with the TReP-132 protein or with a complex comprising the TReP-132 protein, and with a receptor of one or more steroid hormones; and measuring the binding of the TReP-132 protein or of the complex comprising the TReP-132 protein to the receptor of one or more steroid hormones.

In the spirit of the invention, the expression “compound increasing, promoting or facilitating the binding of the TReP-132 protein to the receptor of one or more hormones” denotes any compound, agent, factor, condition or treatment which can increase or stimulate, in a cell, the binding of the TReP-132 protein to the hormone receptor of interest. It can be a ligand of a nuclear receptor, preferably of steroid hormones (in particular protesterone or estrogen) or a steroid analog.

Another object of the invention is based on the use of a compound modulating the binding of the TReP-132 protein to the receptor of one or more steroid hormones, in particular to a progesterone receptor or to an estrogen receptor, for preparing a composition intended for the treatment of a proliferative disease. According to the invention, the modulation of binding can consist in an increase or a decrease of binding of the TReP-132 protein to the receptor of one or more steroid hormones such as those indicated earlier.

In a preferred manner, the invention relates to the use of a compound increasing or promoting the binding of the TReP-132 protein or of the complex comprising said protein to the progesterone receptor and/or decreasing the binding of the TReP-132 protein or of the complex comprising said protein to the estrogen receptor, for preparing a composition intended for the treatment of a proliferative disease and in particular a hormone-dependent cancer.

Another object of the invention is based on the use of a compound increasing or mimicking the activity of the TReP-132 protein for preparing a composition intended for the treatment of a proliferative disease. In particular, the invention can be used in the treatment of hormone-dependent cancers, in particular in the treatment of cancers sensitive to a deregulation of the synthesis and/or activity of progesterone or estrogens such as breast or uterine cancer.

In a preferred embodiment of the invention, the composition is used in combination with a hormone for a simultaneous, separate or sequential administration.

In the spirit of the invention, the expression “compound increasing or mimicking the activity of the TReP-132 protein” denotes any compound, agent, factor, condition or treatment which can increase, mimic or stimulate, in a cell, the activity of the TReP-132 protein.

Preferably they are substances, selected for example in the group consisting of a nucleic acid, a polypeptide, a lipid, a small molecule, and the like. Said compound can be identified with the aid of a method according to the invention.

The compound which is used is preferably a compound which mimics the activity of the TReP-132 protein, stimulates the expression or nuclear transport of the TReP-132 protein and/or increases the concentration of the TReP-132 protein in cells.

Particularly preferred compounds are those which can selectively increase the activity of the TReP-132 protein, that is to say, essentially without directly and significantly affecting the activity of another metabolic pathway.

More specifically, in the spirit of the invention, the term “activity of the TReP-132 protein” refers in particular to the synthesis of said protein (transcription, translation, etc.), maturation thereof, transport thereof to the nucleus, interaction thereof with a receptor or with a nucleic acid, the transcriptional activity thereof, the degradation thereof, etc. The compound increasing the activity of the TReP-132 protein can therefore be an agent increasing the synthesis of the TReP-132 protein, an agent increasing the transport thereof, an agonist of the TReP-132 protein, an agent mimicking the activity of the TReP-132 protein, etc.

In a particular embodiment of the invention, a compound which can increase the synthesis of the TReP-132 protein, that is to say, in particular the transcription or translation of its gene or RNA, is used. Advantageously, it is possible to use a compound which increases the expression of the TReP-132 protein in a cell. One object of the invention is therefore based on the use of a compound increasing the expression of the TReP-132 protein for preparing a composition intended for the treatment of proliferative diseases.

Said compound is preferably a nucleic acid coding for a TReP-132 protein, a compound stimulating the promoter of the TReP-132 gene or a compound increasing the stability of TReP-132 mRNA.

In this regard, a particular object of the invention is based on the use of a nucleic acid coding for a TReP-132 protein for preparing a compound intended for the treatment of a proliferative disease. Another object of the invention is based on a pharmaceutical composition comprising a nucleic acid coding for the TReP-132 protein and a pharmaceutically acceptable excipient.

In the spirit of the invention, the nucleic acid can be a DNA or RNA, for example a genomic, complementary or synthetic DNA, a messenger RNA, etc. Preferably it is a cDNA. A particular nucleic acid codes for a protein corresponding to sequence SEQ ID NO: 2 or for a natural or functional variant of same. More particularly it is any nucleic acid coding for a TReP-132 protein and which can hybridize with sequence SEQ ID NO: 1 or with a region thereof (typically comprising at least 50 bases), in conditions of moderate or high stringency. Suitable stringency conditions are for example an incubation at 42° C. for 12 hours in a solution comprising 50% formamide, 5×SSPE, 5×Denhardt's, 0.1% SDS, (1×SSPE is composed of 0.15 M NaCl, 10 mM NaH₂PO₄, 1.3 mM EDTA, pH 7.4). Of course it is possible to vary the temperature and salt concentration of the medium. In the context of the invention, the nucleic acid can optionally comprise regulatory regions, in particular, a promoter region, poly A, etc. In particular it is possible to use homologous or heterologous promoters, strong or weak, regulated, constitutive or inducible, tissue-specific or ubiquitous, etc. They can be of different origins, such as viral, cellular, bacterial, artificial promoters, etc. Specific examples of promoters include in particular the HSV-LTR, CMV, TK, SV40, PGK, albumin promoter, etc.

The nucleic acid can be cloned in a vector, in particular an expression vector, such as for example a plasmid, cosmid, phage, virus, artificial chromosome, etc. Preferably it is a plasmid vector or a vector derived from a virus, such as for example a retrovirus, adenovirus, AAV, herpes virus, etc. Particularly useful viruses are retroviruses, adenoviruses or AAV, whose genome has been modified to contain a region coding for a TReP-132 protein, and so as to be replication-defective. The methods for producing such defective viruses are known to those skilled in the art, and comprise, for example, introducing a recombinant viral vector in a packaging cell, optionally in the presence of a helper virus or plasmid. The recombinant viruses used in the context of the invention are advantageously replication-defective, which is to say that they are essentially incapable of autonomously replicating in a cell. Said recombinant viruses therefore have a recombinant genome in which one or more viral genes (or viral regions) essential for replication have been disabled (e.g., mutated or deleted, in whole or in part), such as for example the E1 or E4 regions (for adenoviruses), Rep or Cap (for MVs), GAG and/or POL (for retroviruses), etc. Methods for producing recombinant retroviruses are described for example in WO90/02806, U.S. Pat. No. 5,324,645 and WO94/19478. Methods for producing recombinant adenoviruses are described for example in WO95/02697 and WO96/22378.

The nucleic acid or vector can be used in the context of the invention to increase the expression and therefore the activity of the TReP-132 protein in vitro, ex vivo or directly in vivo. More generally it is a direct use in vivo, in the proliferative cells, or ex vivo, from a sample or biopsy of the tissue to be treated, which is then reinjected into the patient, typically after irradiation.

Another particular object of the invention is based on the use of a compound stimulating the TReP-132 gene promoter or increasing the stability of TReP-132 mRNA for preparing a composition intended for the treatment of a proliferative disease. Said compound in fact makes it possible to increase the amount of TReP-132 protein in a cell.

Another object of the invention is based on a pharmaceutical composition, characterized in that it comprises at least one compound stimulating the activity of the TReP-132 gene promoter and a pharmaceutically acceptable excipient. Said compound can be identified, validated or optimized for example with the aid of a method comprising determining the ability of a test compound to bind to the TReP-132 gene promoter or a region thereof, and/or to activate said promoter. Said activity can be determined by measuring the expression of a gene placed under the control of the TReP-132 promoter.

According to another preferred embodiment, the invention makes use of one or more compounds which can increase, induce or stimulate the maturation, stability or activity of the TReP-132 protein in a cell. An object of the invention is therefore based on a compound increasing the maturation, stability or activity of the TReP-132 protein for preparing a composition intended for the treatment of a proliferative disease. The TReP-132 protein and analogs thereof are examples of compounds which can be used.

In fact, according to a preferred embodiment of the invention, it is possible to directly use the TReP-132 protein, typically produced by in vitro recombination, so as to increase the antiproliferative activity. The TReP-132 protein can be produced in vitro by expressing a recombinant nucleic acid in any suitable cell system, and recovering the protein produced. In particular the cell system is a bacteria (e.g., E. coli), yeast (e.g., Saccharomyces, Kluyveromyces), a eukaryotic cell, particularly human (fibroblast, hepatocyte, etc.).

According to another preferred embodiment of the invention, the compound which can increase the transcriptional activity of the TReP-132 protein is represented by any compound increasing the binding of the TReP-132 protein or of a complex comprising the TReP-132 protein to the promoter of a target gene of the TReP-132 protein. Target gene of the TReP-132 protein should be understood to mean any gene whose transcription is regulated by the TReP-132 protein, in particular the p27, p21 and p16 genes. In fact, the application shows that the TReP-132 protein can stimulate the expression of the p27, p21 and p16 genes. The invention therefore discloses a novel pathway and novel molecular targets, which can be used in the scope of efficient therapeutic approaches.

An object of the invention is therefore based on the use of a compound increasing the binding of the TReP-132 protein or of a complex comprising the TReP-132 protein to the promoter of a target gene of the TReP-132 protein for preparing a pharmaceutical composition intended for the treatment of a proliferative disease.

In a preferred embodiment of the invention, a compound is used which increases the activity of the promoter of a target gene of the TReP-132 protein.

In the context of the invention, the target genes of the TReP-132 protein are in particular the genes coding for p21, p27 or p16. Said compound can be selected, validated or optimized by different approaches, and in particular by means of a binding assay or a transcriptional assay employing a reporter gene placed under the control of a promoter having all or part of the sequence of the p27, p21 or p16 gene promoter. Said compound can be a peptide derived from the TReP-132 protein, an antibody, a small molecule, etc.

While any approach allowing to increase or mimic the activity of the TReP-132 protein can be used in the scope of the invention, more particularly preferred are strategies, molecules and conditions allowing to increase the synthesis of the TReP-132 protein, in particular the use of a nucleic acid coding for the TReP-132 protein. In this regard, a particular object of the invention is based on the use of a nucleic acid coding for the TReP-132 protein for preparing a composition intended for the treatment of a proliferative disease.

Another particular object of the invention is based on the use of a compound modulating the formation or activity of the complex comprising the TReP-132 protein, Sp1 and a receptor of one or more steroid hormones, e.g., a progesterone receptor or an estrogen receptor, for preparing a composition intended for the treatment of a proliferative disease, in particular a hormone-dependent cancer.

Preferably, the test compound modulates the interaction of the TReP-132 protein, Sp1 and a receptor of one or more hormones (in particular steroids, e.g., a progesterone receptor or an estrogen receptor), modulates the binding of the complex comprising the TReP-132 protein, Sp1 and said receptor to the promoters of TReP-132 target genes or modulates the action of the complex comprising the TReP-132 protein, Sp1 and a receptor such as described hereinabove on the promoter of a target gene. In the context of the invention, the target genes are in particular the p21, p27 and/or p16 genes.

The invention can be used in the treatment of a proliferative disease, such as cancer. Preferably, the invention is used for treating hormone-dependent cancers, in particular cancers sensitive to a deregulation of the synthesis and/or activity of progesterone or estradiol, even more preferably, breast or uterine cancer.

Use of the Compounds

Considering the physiological functional properties of the target used, the inventive compounds can be used in the treatment of a proliferative disease and in particular in the treatment of a cancer. In particular, the compounds according to the invention are compounds which can mimic or increase the activity of the TReP-132 protein, compounds which can modulate the binding of the TReP-132 protein or of a complex comprising the TReP-132 protein to the receptor of one or more steroid hormones, e.g., a progesterone receptor or an estrogen receptor, or compounds which can modulate the formation or activity of the complex comprising the TReP-132 protein, Sp1 and the receptor of one or more steroid hormones, e.g., a progesterone or an estrogen receptor. In particular, the inventive compounds are compounds identified with the aid of a method such as described hereinabove.

In the spirit of the invention, the term “treatment” refers to preventive, curative, palliative treatment, as well as management of patients (alleviating suffering, prolonging survival, improving quality of life, slowing disease progression, reducing tumor size), etc. Furthermore, the treatment can be carried out in combination with other agents or treatments, in particular addressing different metabolic pathways. It can also be combined with hormonal therapy, chemotherapy or radiotherapy, for example. A particular combined treatment comprises the sequential, simultaneous or separate use of a compound increasing or mimicking the activity of the TReP-132 protein and a hormone. Another particular combined treatment involves the sequential, simultaneous or separate use of a compound stimulating the binding of the TReP-132 protein or of a complex comprising the TReP-132 protein to the receptor of one or more steroid hormones, e.g., to the progesterone receptor or to the estrogen receptor, and a hormone.

A particular object of the invention thus relates to the use of a composition according to the invention for treating a proliferative disease in combination with a hormone for a simultaneous, separate or sequential administration.

The invention can be used for the treatment of proliferative diseases affecting mammals, and humans in particular. The data presented in the examples demonstrate the ability of the TReP-132 protein to inhibit cell proliferation, in particular that of tumor cells, and to block cell cycle progression.

In the spirit of the invention, proliferative disease is understood to mean any pathology characterized by or associated with cell cycle deregulation and/or uncontrolled or pathological cell proliferation. Typical examples of such pathologies are in particular cancers, stenosis, fibrosis, psoriasis, etc. The invention is particularly adapted to the treatment of cancer, and in particular solid tumors, primary or metastasized. In particular these include cancers of the lung, liver, colon, head-and-neck, brain, bladder, spleen, skin, breast, prostate, uterus, thymus, etc. In particular they are hormone-dependent cancers and in particular typically female cancers represented by breast and uterine cancer.

An object of the invention is based on the use of a compound such as defined hereinabove for preparing a composition intended for the treatment of a proliferative disease, in particular for the treatment of a cancer. The invention also discloses methods for treating proliferative diseases based on the use of the aforementioned compounds.

The invention relates in particular to the use of a compound such as defined hereinabove for reducing the proliferation of tumor cells in a patient, and to a corresponding method.

The invention also relates to the use of a compound such as defined hereinabove for reducing tumor progression, or for inducing shrinkage of a tumor in a patient, and to a corresponding method.

The invention further relates to the use of a compound such as defined hereinabove for inducing apoptosis of tumor cells in a patient, and to a corresponding method

Screening Tests

The invention relates to methods for the selection, identification, characterization or optimization of active compounds modulating, preferably reducing, cell proliferation, based on measuring the modulation of binding or activity of the TReP-132 protein or of a complex comprising the TReP-132 protein to a hormone receptor or target gene, measuring the modulation of binding or activity of the complex comprising the TReP-132 protein and a hormone receptor on the promoter of a target gene or measuring the modulation of binding of the complex comprising the TReP-132 protein, a hormone receptor (e.g., a progesterone or estrogen receptor) and its hormone and the Sp1 transcription factor to the promoter of a target gene.

In particular the invention relates to methods for the selection, identification, characterization or optimization of active compounds modulating cell proliferation, comprising determining the ability of a test compound to modulate, i.e. mimic, increase or promote the binding of the TReP-132 protein or of a complex comprising the TReP-132 protein to the receptor of one or more steroid hormones, e.g., to the progesterone receptor or to the estrogen receptor, said receptor optionally being complexed with the Sp1 transcription factor.

In this regard, the screening tests according to the invention are based, in general, on selecting compounds which can increase or promote the binding of the TReP-132 protein or of a complex comprising the TReP-132 protein to the receptor of one or more steroid hormones or else reduce or inhibit the binding of the TReP-132 protein or of a complex comprising the TReP-132 protein to the receptor of one or more steroid hormones.

Another object of the invention relates to methods for the selection, identification, characterization or optimization of active compounds, comprising determining the ability of a test compound to modulate the action of the TReP-132 protein on the promoter of a target gene of the TReP-132 protein.

The screening tests according to the invention are based, in general, on selecting compounds which can modulate the expression of target genes of the TReP-132 protein (expression screening) or on selecting compounds which can modulate the binding of the TReP-132 protein to one or more of its target genes (binding assay), that is to say, genes whose expression is regulated by said protein.

The invention thus relates to a screening test for compounds modulating the formation or activity of the complex comprising a receptor of one or more steroid hormones such as progesterone or estrogens and optionally the TReP-132 protein and/or the Sp1 transcription factor. In this regard, the screening tests according to the invention are based on selecting compounds which can modulate the expression of one or more target genes of the complex comprising a receptor of one or more steroid hormones, and optionally the TReP-132 protein and/or the Sp1 transcription factor.

In the context of the invention, the target genes of the TReP-132 protein are in particular the p21, p27 and p16 genes.

Another aspect of the invention relates to methods for the selection, identification, characterization or optimization of active compounds, comprising measuring the expression of a reporter gene placed under the control of all or part of the promoter sequence of the TReP-132 gene, in the presence of a test compound.

More particularly, the invention provides screening methods for selecting, identifying or characterizing biologically active compounds or methods which can be used for validating, characterizing, optimizing or producing compounds.

The inventive methods can be implemented in different ways, in cellular or acellular tests in vitro, or in vivo.

A—Methods Based on a Binding Assay

An object of the invention is based on a method for the selection, identification, characterization or optimization of active compounds, comprising determining the ability of a test compound to modulate the binding of the TReP-132 protein or of a complex comprising the TReP-132 protein to the receptor of one or more steroid hormones. Preferably, the method comprises determining the ability of a test compound to reduce or inhibit the binding of the TReP-132 protein or of a complex comprising the TReP-132 protein to the receptor of one or more steroid hormones. Even more preferably, the method comprises determining the ability of a test compound to mimic, increase or promote the binding of the TReP-132 protein or of a complex comprising the TReP-132 protein to the receptor of one or more steroid hormones.

In this embodiment, one object of the invention relates to methods for the selection, identification, characterization or optimization of active compounds modulating cell proliferation, comprising:
a) contacting a test compound with:
   i) the TReP-132 protein or complex comprising the TReP-132 protein, and
   ii) the receptor of one or more steroid hormones, and
b) measuring the binding of the TReP-132 protein or of the complex comprising the TReP-132 protein to said receptor.

In the context of the invention, the steroid hormone receptor is an estrogen receptor (ER) or a progesterone receptor (PR), preferably the progesterone receptor (PR).

Another object of the invention relates to a method for the selection, identification, characterization or optimization, in vitro or ex vivo, of active compounds reducing cell proliferation, comprising
a) contacting, in the presence of progesterone or estrogens, a test compound with:
i) respectively, the progesterone or estrogen receptor,
ii) Sp1, and
iii) a nucleic acid comprising all or part of the promoter of a target gene of progesterone or estrogens, and
b) measuring the binding of Sp1 or of the complex comprising the progesterone or estrogen receptor and Sp1 to said promoter, or measuring the expression of said promoter, said measurement being an indication of the effect of the test compound on cell proliferation.

Contact is optionally carried out in the presence of the TReP-132 protein or of a complex comprising the TReP-132 protein. Preferably, contact is carried out in a cell.

As indicated earlier, the steroid hormone target gene is advantageously selected in the group consisting of the p21, p16 and/or p27 genes.

Another object of the invention comprises a method for the selection, identification, characterization or optimization of active compounds comprising determining the ability of a test compound to modulate the binding of the TReP-132 protein or of a complex comprising the TReP-132 protein to the promoter of target genes of the TReP-132 protein, in particular the p27, p21 and p16 genes.

In another embodiment, the method comprises:

a) contacting the test compound in the presence of TReP-132 with a nucleic acid construct comprising a sequence of all or part of a p21, p27 or p16 gene promoter or a region thereof comprising the binding site of the TReP-132 protein or a response element of the TReP-132 protein, and

b) determining the binding of said test compound to the target nucleic acid and/or to the complex formed by the binding of TReP-132 to its response element(s) or binding site(s).

In order to carry out the binding assay, it is possible to use a nucleic acid comprising all or part of the promoter sequence, and to determine in vitro the ability of a test compound to bind thereto. The sequence fragment preferably contains at least 10 consecutive nucleotides of the promoter sequence, more preferably at least 20 consecutive nucleotides of the promoter sequence. Moreover, it is possible to test several fragments of the promoter sequence concomitantly.

The ability of said test compound to modulate the binding of TReP-132 to the receptor of one or more steroid hormones or to the response element of a target gene of the TReP-132 protein can be measured by determining the amount of TReP-132 bound in the presence of the test compound compared to said amount in the absence of the test compound or by carrying out the reaction in the presence of labelled TReP-132 protein and measuring the extent to which the test compound displaces the binding of the labelled protein.

Said tests can be carried out in vitro, in any suitable device (tube, dish, flask, etc.). Optionally they can be carried out with one of the partners being immobilized, for example the nucleic acid (column, bead, support, glass, filter, membrane, etc.).

The binding of the test compound in the context of the inventive methods can be visualized by gel migration, electrophoresis and by other methods based on luminescence or using FRET (Fluorescence Resonance Energy Transfer) or SPA (Scientillation Proximity Assay), or by any other method known to those skilled in the art.

B—Methods Based on Expression Screening

Another object of the invention is based on the transcriptional activity of the TReP-132 protein (transcriptional activity assay).

According to a first preferred embodiment of the invention, the method for the selection, identification or optimization of active compounds comprises:

a) contacting a test compound with a reporter nucleic acid comprising a reporter gene placed under the control of a promoter comprising all or part of the TReP-132 promoter sequence, and

b) measuring the expression of the reporter gene.

Another object of the invention comprises determining the ability of a test compound to modulate the expression of one or more target genes of the TReP-132 protein, in particular the p21, p16 and/or p27 genes.

In this embodiment, the method comprises:

a) contacting a test compound, in the presence of the TReP-132 protein, with a reporter nucleic acid comprising a reporter gene placed under the control of a promoter containing all or part of the sequence of the p21, p16 or p27 gene promoter or the binding site of the TReP-132 protein or a response element of the TReP-132 protein, and

b) determining the effect of the test compound on the expression of the reporter gene.

In a particular embodiment of the inventive methods, the possible effects obtained are compared with the possible effects determined by a method carried out under the same conditions but with a nucleic acid construct comprising at least one inactive variant (for example a mutant copy) of the p27, p21 or p16 promoter sequence or a region thereof comprising the binding site of the TReP-132 protein or a TReP-132 response element.

Studies have shown that TReP-132 can directly interact with DNA to regulate promoter activity. Furthermore, it has also been shown that TReP-132 interacts with other DNA-binding proteins to regulate transcription. Thus, TReP-132 can function as a coregulator of nuclear receptors like SF-1 to regulate gene expression. For this reason, TReP-132 can also regulate promoter activity without interacting with DNA.

Considering that TReP-132 can function as a coregulator of nuclear receptors, the whole protein or fragments thereof can be used in a protein recruitment assay requiring the presence of a ligand and for screening ligands of partner nuclear receptors. The protein corresponding to a nuclear receptor X can be incubated with the whole TReP-132 protein or with fragments thereof, so as to enable recruitment of the TReP-132 polypeptide by the nuclear receptor, in the presence of the ligand. Depending on the type of labelling of the nuclear receptor and the TReP-132 polypeptide, different methods can be employed to measure protein-protein interaction or protein recruitment, including fluorescence, scintillation counting or calorimetry. The isolated compounds can lead to an increase or decrease in TReP-132 recruitment in the mechanism of expression of the gene. By using said approaches, it is possible to screen compounds binding nuclear receptors which affect the expression of target genes regulated by TReP-132.

A particular object of the invention relates to a method for the selection, identification, characterization or optimization of active compounds comprising determining the ability of a test compound to modulate the activity of the complex composed of at least the TReP-132 protein and a receptor of one or more steroid hormones.

Another object of the invention relates to a method for the selection, identification, characterization or optimization of active compounds comprising determining the ability of a test compound to modulate the expression of target genes of the complex comprising the TReP-132 protein and a receptor of one or more steroid hormones.

In this particular embodiment of the invention, the method comprises:

a) contacting the test compound with the TReP-132 protein and a reporter nucleic acid comprising a reporter gene placed under the control of a promoter comprising the promoter sequence of a target gene of the complex comprising the TReP-132 protein and a receptor of one or more steroid hormones, or a region of said promoter containing the binding site of the complex or a response element of the complex, and

b) measuring the expression of said reporter gene.

In the context of the invention, the receptor of one or more steroid hormones is an estrogen receptor (ER) or a progesterone receptor (PR).

The target genes of the TReP-132 protein are in particular the p27, p21 or p16 genes.

Another object of the invention relates to a method for the selection, identification, characterization or optimization, in vitro or ex vivo, of active compounds reducing cell proliferation, comprising:

a) contacting, in the presence of progesterone or estrogens, a test compound with:

i) respectively, the progesterone or estrogen receptor,
ii) Sp1, and
iii) a nucleic acid comprising all or part of the promoter of a progesterone or estrogen target gene, and
   b) measuring the binding of Sp1 or of the complex comprising the progesterone or estrogen receptor and Sp1 to said promoter, or measuring the expression of said promoter, said measurement being an indication of the effect of the test compound on cell proliferation.

The inventive methods can be carried out with different types of cells, promoters, reporter genes, and in different conditions, as described hereinbelow.

Host Cell:

Some screening methods, used in the context of the invention, comprise a step of contacting the test compound with a host cell, in specific conditions allowing to determine the expression in said cell of a reporter gene or of the TReP-132 protein, to possibly reveal other steps in the synthesis of the TReP-132 protein, and thereby to obtain information about the effect of the test compound. Classically, the effect of the test compound is compared with the level of reporter gene expression or the activity of the expression product determined in the absence of said compound.

The cells which are used can be selected from among any cells that can be cultured in the laboratory. In a preferred embodiment of the invention, they are mammalian cells (hepatocytes, fibroblasts, endothelial, muscle, breast cells, etc.). Even more preferably, said cells are of human origin. They can be primary cultures or established cell lines. In another embodiment, it is also possible to use prokaryotic cells (bacteria), yeast cells (Saccharomyces, Kluyveromyces, etc.), plant cells, etc.

Reporter System:

A particular embodiment of the invention makes use of a reporter system comprising a reporter gene placed under the control of a particular promoter. Said construct, or any cassette or vector containing it, can be introduced into host cells, which can be used for cellular tests.

To carry out the transcriptional assay, a reporter system comprising all or part of the promoter of the target gene operationally linked to a reporter gene is advantageously used.

Said reporter gene can be in particular any gene whose transcription or expression product can be detected or measured in biological extracts. For example, it can be the gene coding for luciferase and more particular for firefly or Renilla luciferase, for secreted alkaline phosphatase, galactosidase, lactamase, chloramphenicol acetyl transferase (CAT), human growth hormone (hGH), β-glucuronidase (Gluc) and green fluorescent protein (GFP), etc. It shall be understood that the term “gene” denotes in the broad sense any nucleic acid, in particular a cDNA, gDNA, synthetic DNA, an RNA, etc.

The reporter gene, whatever it may be, is placed under the control of a promoter containing all or part of the promoter sequence of a target gene of the TReP-132 protein or of the complex comprising the TReP-132 protein and a receptor of one or more steroid hormones, the promoter of the gene coding for the TReP-132 protein, the promoter of the complex comprising the TReP-132 protein, Sp1 and a receptor of one or more steroid hormones or a functional variant thereof, such as defined hereinabove. Preferably, it is a promoter whose differential activity in the presence and absence of TReP-132 or a functional equivalent thereof can be detected.

In a preferred embodiment of the invention, the reporter gene is placed under the control of a promoter comprising the complete promoter sequence of a target gene of the TReP-132 protein, a target gene of the complex comprising the TReP-132 protein and a receptor of one or more steroid hormones, the promoter of the complex comprising the TReP-132 protein, Sp1 and a receptor of one or more steroid hormones or the promoter of the gene coding for the TReP-132 protein. In this regard, an object of the invention also relates to a nucleic acid comprising a reporter gene placed under the control of a promoter comprising all or part of the p27, p21 or p16 promoter sequence, in particular the human promoter. The optionally selected promoter fragment advantageously comprises at least 10 consecutive nucleotides of said sequence, preferably at least 20, more preferably at least 30, even more preferably at least 50. The promoter can also comprise heterologous regions, originating from other genes or promoters, such as for example silencer or enhancer signals, sequences conferring regulated or tissue-specific features, etc. Said particular sequences can be present in one or more copies in the promoter (preferably 1 to 10 and more preferably 1 to 6), upstream, downstream, or internally, in the same orientation or in the opposite orientation. The promoter can be a hybrid promoter combining regions from other promoters, for example the promoter of the herpes virus thymidine kinase (TK) gene, the CMV early promoter, the PGK promoter, the SV40 promoter, etc.

Furthermore, the different aforementioned functional domains can directly flank each other, or be separated by nucleotides which do not significantly affect the functionality of the expression cassette or which give the system improved performance or characteristics (amplifier, silencer, intron, splicing site, etc.).

The construct can be cloned in any suitable vector, such as a plasmid, cosmid, phage, virus, etc. The construct or vector can be introduced into a host cell by any conventional method, including in particular electroporation, calcium phosphate precipitation, liposomes, transfectants, etc. The cells or the descendents thereof can be cultured in any suitable medium (DMEM, RPMI, etc.).

Contact:

The test compounds can be contacted with the cells at different times, according to their effect(s), their concentration, the nature of the cells and technical considerations.

Contact can be carried out on any suitable support and in particular on a plate, dish, in a tube or flask. Generally, contact is carried out on a multiwell plate which allows many different tests to be carried out at the same time. Typical supports include microtitration plates and more particularly plates with 96 or 384 wells (or more), which are easy to manipulate.

Depending on the support and the nature of the test compound, variable amounts of cells can be used to carry out the aforementioned methods. Classically, between 10³ and 10⁶ cells are contacted with a particular test compound, in a suitable culture medium, and preferably between 10⁴ and 10⁵ cells. As an example, in a 96-well plate, 10⁵ cells can be incubated in each well with a desired quantity of a test compound; in a 384-well plate, fewer than 10⁵ cells and typically between 1×10⁴ and 4×10⁴ cells are generally incubated in each well with the test compound.

The quantity (or the concentration) of test compound can be adjusted by the user according to the characteristics of said compound (its toxicity, ability to penetrate cells, etc.), the number of cells, the length of the incubation period, etc. Generally, the cells are exposed to concentrations of test compounds ranging from 1 nM to 1 mM. Of course it is possible to test other concentrations without deviating from the invention.

Also, each compound can be tested in parallel at different concentrations.

Different adjuvants and/or vectors and/or products facilitating the penetration of the compounds into the cells such as liposomes, cationic lipids or polymers can also be used, when necessary.

Contact is typically maintained for several minutes to several hours, generally between 1 and 48 hours. In particular, when the assay comprises the expression of a reporter gene, the cells and various reagents should preferably remain in contact long enough to allow de novo synthesis of the reporter gene expression product.

Measurement of Effect:

The measurement or demonstration of an effect of the test compound can be carried out in different ways, according to the assay used.

Methods for detection of binding in vitro are mentioned hereinabove.

Transcriptional activity assays comprise a step of determining and detecting the expression of the reporter gene.

This may be a determination of transcriptional activity. To this end, total RNA is extracted from cultured cells in the experimental conditions on the one hand and in a control situation on the other hand. Said RNA is assayed (or used as probe) to analyze changes in expression of the reporter gene(s).

It can also be a matter of visualizing or assaying the reporter gene expression product. Said visualization (or said assay) can be achieved by different methods the nature of which depends on the type of reporter gene used. For example, the measurement can correspond to an optical density or a fluorescence emission in the case where the β-galactosidase or luciferase gene is used as reporter gene.

The expression of the reporter gene can also be measured in terms of the hydrolysis of a substrate of the reporter gene expression product, such as for example a substrate of β-lactamase. One can mention in particuar any product containing a β-lactam nucleus and the hydrolysis of which can be measured. Preferred substrates are those specific of β-lactamase (i.e., they are generally not hydrolyzed in mammalian cells in the absence of β-lactamase), those which are nontoxic to mammalian cells and/or whose hydrolysis product can be easily measured, for example by methods based on fluorescence, radioactivity, an enzymatic activity or any other method of detection.

Even more preferred substrates are the radiometric substrates. The hydrolysis of said substrates can be directly correlated with the activity of the reporter gene product by the number of cells.

The expression product can also be measured by immunological or immunoenzymatic methods, involving a specific antibody for example. Said system is particularly suited to assaying for example the TReP-132 protein synthesized by a cell treated or not treated with a test compound.

In a general manner, the presence of the reporter gene product (or the hydrolysis product of the substrate) can be determined by conventional methods known to those skilled in the art [fluorescence, radioactivity, OD, luminescence, FRET (see WO 0037077), SPA, biochips, immunological methods, etc.]. Generally, one determines the activity of a test compound in a cell and said effect is compared with the level of activity in the absence of test compound or with a mean value determined in the absence of any test compound.

Hydrolysis is measured essentially by measuring (or determining the relative amount) of the hydrolysis product contained in each reaction sample with the aid of different methods known to those skilled in the art, including detection of fluorescence, radioactivity, color, an enzymatic activity, an antibody-antigen immune complex.

A secondary test can be carried out whereby the selection of the compounds can be validated, for example by determining cell proliferation in culture or in animals, or by comparison with untreated cells or animals.

The invention is particularly adapted to the selection, identification or characterization of a large number of compounds. Said simple and efficient screening can be accomplished in a very short time. In particular, the described methods can be partially automated, thereby enabling the efficient and simultaneous screening of many different compounds, either in the form of a mixture or separately.

The aforementioned methods for the selection, identification and characterization of test compounds according to the invention can be used for the selection, identification and characterization of compounds which can inhibit cell proliferation and/or regulate the cell cycle.

Other objects of the invention relate to a method for detecting a predisposition, a method of diagnosis, a method of monitoring and measuring the aggressiveness of a proliferative disease comprising measuring the binding of the TReP-132 protein to one or more steroid hormones in a sample taken from a mammal, in particular a human. As indicated earlier, the receptor of one or more steroid hormones is preferably a progesterone receptor (PR) or an estrogen receptor (ER).

The aforementioned methods can comprise or involve solely the assay of the TReP-132 protein.

A particular object of the invention further relates to a method for detecting a predisposition, a method of diagnosis, monitoring and measuring the aggressiveness of a proliferative disease comprising detecting, in a patient, the overexpression of estradiol, the reduction of progesterone or a decrease in the expression of the CDKIs p21, p16 and/or p27.

Test Compounds

The compounds which can be identified with the aid of a method according to the invention can be compounds of different nature, structure and origin, in particular biological compounds, nuclear factors, cofactors, chemical, synthetic compounds, etc. The invention can therefore be used with any type of test compound. For instance, the test compound can be any product which is isolated or in a mixture with other products. The compound can be defined in terms of structure and/or composition or be defined in terms of function. For example, the compound can be an isolated and structurally defined product, an isolated product with undefined structure, a mixture of known and characterized products or an undefined composition comprising one or more products. Hence one or more compounds can be tested, in a mixture or separately. For example, said undefined compositions can be samples of tissues, biological fluids, cell supernatants, plant preparations, and the like. The test compounds can be selected from among an inorganic or organic product and in particular a polypeptide; protein or peptide, a nucleic acid, lipid, polysaccharide and a chemical or biological compound. It can be for example a nuclear factor, a cofactor or any mixture or derivative thereof. The compound can be natural or synthetic and can include a combinatorial library, a clone or a library of nucleic acid clones expressing one or more DNA-binding polypeptides, etc.

Pharmaceutical Composition

A particular object of the invention relates to a pharmaceutical composition comprising a compound increasing or mimicking the activity of the TReP-132 protein and a pharmaceutically acceptable excipient.

Preferably, said pharmaceutical composition is used in combination with a hormone for a simultaneous, separate or sequential administration.

Even more preferably, the pharmaceutical composition according to the invention comprises the TReP-132 protein or an analog thereof in a pharmaceutically acceptable excipient.

The invention relates to a pharmaceutical composition comprising a compound modulating, in particular mimicking, increasing or promoting, the binding of the TReP-132 protein or of a complex comprising the TReP-132 protein to the receptor of one or more steroid hormones and a pharmaceutically acceptable excipient.

Preferably, the pharmaceutical composition comprises a compound stimulating the binding of the TReP-132 protein to the receptor of one or more steroid hormones and a pharmaceutically acceptable excipient. Even more preferably, the pharmaceutical composition comprises a compound increasing the binding of the TReP-132 protein to the progesterone receptor (PR) or to the estrogen receptor (ER).

Another object of the invention relates to a pharmaceutical composition characterized in that it comprises, in a pharmaceutically acceptable vehicle, a compound stimulating the activity of the complex comprising the TReP-132 protein and a receptor of one or more steroid hormones.

Another particular object of the invention relates to a pharmaceutical composition, characterized in that it comprises, in a pharmaceutically acceptable excipient, a compound modulating, in particular mimicking, increasing or promoting, the action of the TReP-132 protein on one of its target gene promoters, in particular that of the p21, p27 or p16 gene.

The invention also relates to a pharmaceutical composition comprising, in a pharmaceutically acceptable vehicle, a nucleic acid coding for the TReP-132 protein. Preferably, the nucleic acid is cloned in an expression vector, preferably a plasmid or viral vector.

Another object of the invention relates to a pharmaceutical composition comprising, in a pharmaceutically acceptable excipient, a compound modulating the formation or activity of the complex comprising the TReP-132 protein, Sp1 and a receptor of one or more steroid hormones.

A particular object of the invention relates to a pharmaceutical composition comprising, in a pharmaceutically acceptable excipient, a compound selected in the group consisting of the TReP-132 protein, an analog thereof, a compound increasing or mimicking the activity of TReP-132 and a compound identified with the aid of the one of the methods according to the invention, combined with one or more hormones, in view of a simultaneous, separate or sequential administration for the treatment of a proliferative disease.

In a preferred manner, the pharmaceutical composition according to the invention comprises a compound identified with the aid of a method such as described hereinabove. Even more preferably, the selected compound is TReP-132.

In a preferred manner, the hormone combined with the selected compound is progesterone.

The pharmaceutical compositions according to the invention, intended for the treatment of proliferative diseases, can be prepared in particular according to a method comprising a step involving a method such as described earlier.

The pharmaceutical compositions according to the invention advantageously comprise one or more pharmaceutically acceptable excipients or vehicles. Examples include saline, physiological, isotonic, buffered solutions and the like, compatible with pharmaceutical use and known to those skilled in the art. The compositions can contain one or more agents or vehicles selected in the group consisting of dispersants, solubilizers, stabilizers, surfactants, preservatives, and the like. Also, the inventive compositions can comprise other active ingredients or agents.

The compounds or compositions of the invention can be administered in different ways and in different forms. The inventive compounds are typically administered by the systemic route, preferably locally. Advantageously, intratumoral injection as well as injection in a region close to the tumor or irrigating a tumor can be mentioned.

Different doses can be employed, according to the compound, the number of administrations, combination with other active ingredients, the stage of disease, etc. The invention discloses an advantageous and efficient novel strategy for treating proliferative diseases. In fact, the invention is based on the characterization of a natural metabolic pathway which can be subjected to selective interventions.

Other advantages and applications of the invention will become apparent in the following examples, which are given for purposes of illustration and not by way of limitation.

### LEGENDS TO THE FIGURES

FIG. 1: Effect of the TReP-132 protein on formation of G418-resistant colonies.

FIG. 2: Expression of the TReP-132 protein in MCF-7 breast cancer cells, estrogen-dependent.

FIG. 3: Induction of TReP-132 by progesterone and suppression of progesterone inhibition of T47-D cell proliferation following TReP-132 knockdown by using small interfering RNA (“siRNA”). (T47-D cells were treated for the indicated times with 30 nM progesterone). Experiments were carried out in triplicate, and values represent the mean ±SD of a representative experiment.

FIG. 3A: Analysis of TReP-132 mRNA levels by quantitative RT-PCR.

FIG. 3B: Cells transfected with control siRNA or TReP-132 siRNA (400 ng) at 0 and 3 days were recovered at days 2, 4 or 6 and counted. (ns: not significant −*, p<0.05; **, p<0.0; ***, p<0.001).

FIG. 4: Bidirectional Tet-Off expression system.

FIG. 5: Induction of TReP-132 protein expression in the absence of doxycycline.

FIG. 6: Inhibition of cell proliferation by the TReP-132 protein.

FIG. 7: Effet of TReP-132 protein induction on the cell cycle G₁→S transition.

FIG. 8A, 8B: Expression of TReP-132 mRNA after cell synchronization at the end of G₁ phase.

FIG. 9: Effect of TReP-132 on G₁ CDK activities and pRb phosphorylation levels.

FIG. 9A: Kinase activities of each immunoprecipitated cyclin or CDK were quantified using pRb as substrate to measure cyclin D1/CDK6 activity, or histone H1 to measure cyclin A/CDK2 activity. The levels of phosphorylation of histone H1 or pRb in induced HTO cells (HeLa Tet Off) are expressed in relation to the levels in non-induced HTO cells (corresponding to level 1.0).

FIG. 9B: Protein extracts (20 μg) of HTO cells expressing GFP or GFP and TReP-132 under the control of doxycycline, incubated or not with dox for the indicated times, were analyzed by western blot using anti-pRb antibodies directed against all pRb forms (hyperphopshorylated, phosphorylated and hypophosphorylated) or only against pRb phosphorylated at Ser 780, a target residue of cyclin D1/CDK4.

FIG. 10: Effect of induction of TReP-132 protein expression on the transcription of key cell cycle regulatory genes (CDK and CDKI).

FIG. 11: TReP-132 protein activates the p16, p21 and p27 promoters.

FIG. 12: Synergistic action of TReP-132 protein with Sp1 and p300 on p21 promoter activation.

FIG. 13: Induction of p21 and p27 promoter activity by TReP-132 involving an interaction with Sp1 in HeLa cells.

FIGS. 13A and 13B: Luciferase reporter constructs under the control of the deleted or mutant p21 and p27 promoters were cotransfected in HeLa cells with Sp1 (0.1 μg) and/or TReP-132 (0.3 μg) expression vectors. Cell lysates were then assayed for luciferase activity. The results represent the levels of luciferase activity, the activity of the −2320p21 Luc and −3568p27 Luc reporter constructs being arbitrarily set to 1.0. Arrows indicate transcription initiation sites.

FIG. 13C: HTO-GFP or HTO-GFP/TReP-132 cells incubated or not with Dox for 48 hours were recovered for extraction of soluble chromatin. The immunoprecipitations (IPs) were carried out with anti-Flag antibody. Control PCRs were carried out without DNA (H₂O) or with unprecipitated genomic DNA (input). The DNA extracts were amplified using the following primers: region −117/−65 of the p21 gene promoter, region distal to the p21 gene promoter (1 kb upstream of promoter), region −549/−511 of the p27 gene promoter, region distal to the p27 gene promoter (1 kb upstream of promoter) or a region of the beta-actin gene.

FIG. 13D: GST-TReP-132 proteins were incubated with ³⁵-[S]-Sp1 and immobilized on a glutathione-Sepharose column. Specific interactions were analyzed by comparison with the results obtained by incubating GST alone with ³⁵-[S]-Sp1. Sp1 input: 1/10^{th} of radiolabelled Sp1 level used in the incubations.

FIG. 14: Increase in progesterone-dependent activation of the p21 and p27 gene promoters by TReP-132, via increased binding to Sp1 binding sites in T47-D cells.

FIGS. 14A and 14B: T47-D cells were cotransfected with reporter constructs containing the p21 (FIG. 14A) or p27 (FIG. 14B) promoter. Twelve hours post-transfection, the cells were incubated with 30 nM progesterone or ethanol for 34 hours. The cell lysates were then assayed for luciferase activity. Luciferase activity of the −93p21 Luc and −549p27 Luc constructs represent level 1. Arrows indicate transcription initiation sites.

FIG. 14C: Chromatin was from T47-D cells treated or not with 30 mM progesterone. IPs were carried out with anti-TReP-132 antibodies (columns 2-3). Controls were DNA-free PCR products (column 1, H₂O), PCR products of unprecipitated genomic DNA (columns 2 and 3, input), immunoprecipitations with no antibody (columns 4 and 5, no Ab) or with irrelevant antibody (columns 6 and 7, anti-HA). The extracted DNA was amplified using the same primers as in FIG. 13C.

FIG. 14D left: GST-TReP-132 immobilized on glutathione-Sepharose was incubated with ³⁵-[S]-methionine-labelled PR, in the absence or with increasing concentrations of progesterone (0-10 μM). Specific interactions were analyzed by comparison with the results obtained by incubating GST with ³⁵-[S]-PR, in the presence of 100 μM progesterone (GST).

FIG. 14D right: GST-TReP-132 wt, GST-TReP-132 ml (mutated at the LRQLL site) and GST-TReP-132 m2 (mutated at the LEMLL site) were analyzed for interaction with ³⁵-[S]-PR as described earlier, in the presence of 10 μM progesterone. Input PR: 1/10^{th} of the radiolabelled PR level used in the experiments.

FIG. 15: Analysis of the interaction between TReP-132 and the estrogen receptor (ER).

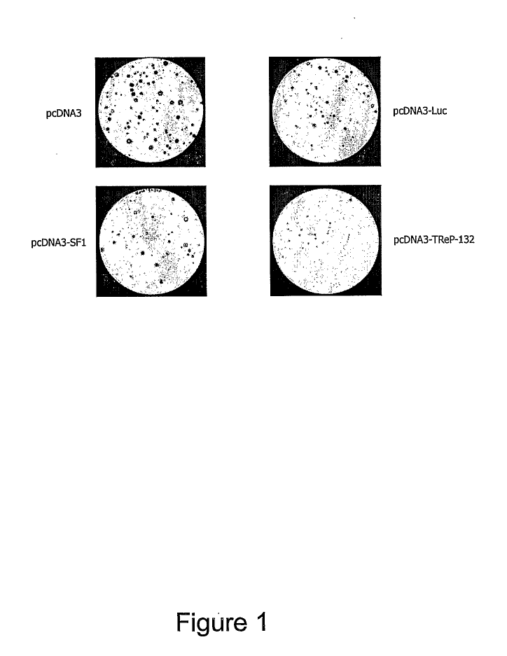

GST-TReP-132 wt was analyzed in terms of the interaction with the ³⁵[S]-ER, as described previously.

### EXAMPLES

### Materials and Methods

1. Plasmids.

The TReP-132-HA vector, containing the entire TReP-132 cDNA tagged with the HA epitope at the 3′ end, was constructed by PCR amplification of TReP-132 cDNA with oligonucleotides introducing a KpnI restriction site at the 5′ end and a HA epitope and XbaI site at the 3′ end. The PCR products were digested with KpnI and XbaI and subcloned into the pcDNA3 expression vector (Invitrogen, Carlsbad, Calif.). The GST-TReP-132 vector was generated by in-frame fusion of the TReP-132 coding region downstream of the GST sequence in the pGEX-2TK vector (Amersham Pharmacia Biotech, Baie d'Urfé, Quebec, Canada). The mutants GST-TReP-132 ml and GST-TReP-132 m2 were constructed using the QuickChange site-directed mutagenesis kit (Stratagene), by mutation of leucines to alanines in NR-boxes (Nuclear Receptor box) 1 and 2 (Gizard et al., 2002b). The pBI-EGFP plasmid which contains a tetracycline-responsive bidirectional promoter enables coexpression of a gene of interest with green fluorescent protein (EGFP for “Enhanced Green Fluorescent Protein”). This plasmid, and the pBI-EGFP-Luc control plasmid expressing both GFP and luciferase, were obtained from Clontech Laboratories, Palo Alto, Calif. The pBI-EGFP-TReP-132 plasmid was formed by directly cloning the entire TReP-132 cDNA obtained by PCR into the NheI site of pBI-EGFP. All constructs were verified by dideoxynucleotide sequencing. The pcDNA3-Sp1 expression vector (Blais et al., 2002) and the human gene p21^{WAF1} reporter plasmid containing the −1560/+34 fragment (p21-Luc −1560/+34) subcloned into the pGL3 vector were generously provided by Dr. Monté (IBL, France). The −2320p21 Luc and −154p21 Luc constructs were generously provided by Drs Kraft and Biggs (Division of Oncology, University of Colorado, Health Sciences Center, Denver), the −93p21 construct Luc and mutants thereof by Dr. Wang (Dept. of Pharmacology, Duke University Medical Center, NC), and the human p27^{KiP1} reporter construct subcloned into the pGL2 basic vector (Promega) by Dr. Toshiyuki Sakai (Kyoto Prefectural University of Medecine, Japan). The 5′ region of the human p16^{CDKN2} gene containing the nucleotide sequence from −1 to −869 relative to the ATG, subcloned between the KpnI and BgIII sites of pGL2 was kindly provided by Dr. Gordon Peters. The series of p16^{CDKN2} reporter vectors were constructed by this team by subcloning different PCR fragments generated with 5′-specific oligonucleotides of the gene.

2. Cell Cultures.

Human cervical HeLa cells and breast tumor cell lines T47-D and MCF-7 were obtained from the ATCC (American Type Culture Collection, Manassas, Va.) and were cultured in monolayers. HeLa cells were grown in DMEM medium (Dulbecco's modified Eagle's medium) (Life Technologies, Gaithersburg, Md.), MCF-7 cells in DMEM-F12 medium (composed of an equal mixture of DMEM and Ham's F-12 medium), and T47-D cells in RPMI 1640. All media were supplemented with 10% fetal calf serum (Hyclone, Logan, Utah), 2 mM glutamine, 50 U/ml penicillin and 50 mg/ml streptomycin (Life Technologies, Ontario, CA). For MCF-7 cells, the medium was supplemented with 10⁻⁹ M estradiol (E2) (Sigma-Aldrich CA Ltd.).

For transfection assays, HeLa and T47-D cells were incubated for 24 hours in 24-well dishes at an initial density of 1.5×10⁴ and 1.5×10⁵ cells per well, respectively. HeLa cells were then transfected with ExGen 500 (MBI Fermentas, Flamborough, Ontario, Canada) at a ratio of 4 μl of ExGen 500 per 0.5 μg of DNA. T47-D cells were transfected with FuGENE 6 (Roche Molecular Biochemicals, Laval, Canada) at a ratio of 3:1 (FuGENE:DNA).

The pGL2 and pGL3 reporter constructs (100 ng) were cotransfected with the indicated amounts of expression vectors and 10 ng of the control plasmid pRL.Null. The amount of plasmid was then normalized with the empty vector pcDNA3. After 12 hours of transfection, the medium was changed and cells were incubated for an additional 10 and 34 hours, respectively. During this period, T47-D cells were cultured in the presence and absence of progesterone (30 nM). The cells were then harvested and cell lysates (20 μl) were assayed for luciferase activity (Dual Luciférase™ Reporter Assay System, Promega, Madison, Wis., USA) in a Berthold LUMAT LB9501 luminometer. The transfections were performed in triplicate and each experiment was repeated at least three times.

3. Colony Formation Assays.

HeLa cells were seeded in 12-well plates at an initial density of 100,000 cells per well, incubated for 24 hours, then transfected with each of the following plasmids: pcDNA3, pcDNA3-Luc, pcDNA3-SF-1, or pcDNA3-TReP-132, each containing the neomycin resistance gene. The transfections were carried out in triplicate with ExGen 500 (MBI Fermentas, Flamborough, Ontario, Canada) at a ratio of 5 μl of ExGen 500 per 1 μg of plasmid. After 12 hours of transfection, the medium was changed and the cells were incubated for an additional 8 hours. The cells were then trypsinized and transferred to a 100 mm petri dish. After 48 hours, the medium was supplemented with 400 μg/ml of the selection agent G418 (Sigma-Aldrich CA Ltd.). Distinct colonies could be detected after two weeks of selection with G418. The cells were then rinsed with PBS and fixed with a mixture of 10% acetic acid/10% methanol for 15 min, then colored with crystal violet (Sigma-Aldrich CA Ltd.).

4. RNase protection assays in MCF-7 cells.

The radiolabelled TReP-132 probe was prepared as follows: the pcDNA3-TReP-132-HA plasmid was first linearized by digestion with the restriction enzyme Narl (New England Biolabs, Beverly, Mass.) before priming in vitro transcription from the T7 promoter in the presence of [α-³²P]UTP ribonucleotide in the conditions specified in the MAXIscript kit (Ambion, Austin, Tex.). This generated a 226 bp cRNA probe containing 174 nucleotide bases located at the 3′ end of TReP-132 and 52 bases originating from the polylinker and including the HA tag. The 18S riboprobe used for normalization was generated from the human pTRI-18S vector (Ambion, Austin, Tex.) and protects an 80 bp fragment. Ribonuclease protection assays were carried out with the Ribonuclease II Protection kit (Ambion, Austin, Tex.) according to the manufacturer's protocol. Thirty micrograms of RNA from MCF-7 cells were hybridized with the two probes for 16 hours at 45° C. and the hybrids were then digested for 45 min at 37° C. in RNase A/RNase T1 solution diluted 1:75 in digestion buffer. The samples were separated on a polyacrylamide-6% urea gel.

5. Reverse transcription and quantitative PCR.

Total RNA from T47-D cells treated or not with progesterone was reverse transcribed and TReP-132 transcript levels were measured by quantitative RT-PCR as described in Gizard et al. (2004) using primers specific for TReP-132 (5′-gtcaacaatatggcccaggtg-3′ and 5′-gccagaggctgctggtcgtc-3′).

6. Silencing of TReP-132 Expression by the Small Interfering RNA (siRNA) Method.

The sense 5′-aacatgtttgagttgccaggcctgtctc-3′ and the antisense 5′-aacctggccaactcaaacatgcctgtctc-3′ oligonucleotides were synthesized (Eurogentec) and used to generate double-stranded siRNA specific for TReP-132 (21 bp corresponding to amino acids 861-881 upstream of the initiation codon of the human TReP-132 gene). The nonsilencing siRNA oligonucleotide from Qiagen, which does not target any mammalian gene, was used as negative control. Transfection was carried out by using the GeneSilencer reagent (Gene Therapy Systems, San Diego, USA) as directed by the manufacturer. Transfection efficiency was verified by FACS analysis (Fluorescence-Activated Cell Sorting) (see below). Quantitative PCR showed a decrease of 75±12% in TReP-132 mRNA levels. Statistical analyses were carried out with the nonparametric Kruskal-Wallis test.

7. Synchronization of HeLa Cells in G₁ phase.

HeLa cells were synchronized in G₁ phase at the G₁→S transition by a double thymidine block as described by Cao et al. (1991) and Tobey et al. (1967) with some modifications. Addition of an excess of thymidine inhibits the enzymatic reaction catalyzing the formation of deoxycytidine triphosphate from cytidine-5′-phosphate and prevents DNA replication, which occurs in the S phase. HeLa cells were first seeded at 10⁶ cells per petri dish (100 mm diameter) and cultured for 24 hours. Thymidine was added at 5 mM final concentration and the cells were incubated for 14 hours. This time interval was calculated to be slightly longer than the sum of the G₂, M and G₁ phases for HeLa cells. The cells were then synchronized to enter S phase by removing thymidine from the medium for 9.5 hours, which is longer than the duration of the S phase. The cell cycle was halted prior to entering S phase by again adding thymidine to the medium for 5 hours. The time at which thymidine was removed corresponds to time point zero in these experiments.

8. Inducible TReP-132 Expression by the Tet-Off System.

HeLa Tet-off cells expressing the tTA transactivator under the control of tetracycline or doxycycline (Dox) (Clontech Laboratories, Palo Alto, Calif.) were stably transfected with the pBI-EGFP-TReP-132 vector or the pBI-EGFP control vector. In order to select stably transfected cells, the pcDNA6 plasmid (Invitrogen, Ontario, CA) containing the blasticidin resistance gene was cotransfected at a ratio of 1:50. For these transfections, the cells were first seeded in 6-well plates at an initial density of 1×10⁵ cells per well, incubated for 24 hours, then transfected with ExGen 500 (MBI Fermentas, Flamborough, Ontario, CA) at a ratio of 5 μl of ExGen 500 for a total of 1 μg of plasmid such as described hereinabove in the section “Colony formation assays”. 48 hours post-transfection, cells were selected for 2 weeks with blasticidin (Sigma-Aldrich CA Ltd.). Clearly visible colonies were then were picked and subcloned on 12-well plates to enable growth. It should be noted that the cells were continuously kept in the presence of doxycycline so as to repress the transcription of genes under control of the tTA transactivator.

9. Cell Sorting by FACS (Fluorescence-Activated Cell Sorting).

To determine the efficiency of the doxycycline-controlled inducible expression system in clones stably transfected with the pBI-EGFP-TReP vector (system described hereinabove), the fluorescence of each cell within a given population was measured individually by flow cytometry on an EPICS XL (Beckman Coulter, Mississauga, CA). The following parameters were measured: FS (Forward scatter), SS (side scatter) and fluorescence emission at 520 nm after excitation with an argon laser at 488 nm. Cells expressing GFP were then sorted on an EPICS ELITE ESP (Beckman Coulter, Mississauga, CA).

10. Measurement of Cell Proliferation.

The cell proliferation rate was determined by the cellular DNA content using the fluorochorome DABA (dimethylaminobenzaldehyde). The medium was removed and cells were dehydrated by addition of 150 μl of methanol to each well. After complete evaporation, cells were incubated for 1 hour at 60° C. with 150 μl of DABA solution prepared as follows: 2 g of DABA were dissolved in 10 ml of 1 N hydrochloric acid (HCl), followed by addition of Norit A Charcoal at 1 g/10 ml. The mixture was shaken, centrifuged for 30 minutes at room temperature, then filtered on a 0.45 micron filter. After incubation, the plates were placed on ice for 10 min and 1.25 ml of 1 N HCl was added to each tube. Fluorescence was measured on a Perkin-Elmer LS-2B Filter Fluorimeter at an excitation wavelength of 400 nm and an emission wavelength of 508 nm. The DNA content was calculated from a standard curve of known quantities of salmon sperm DNA.

11. Cell Cycle Analysis by Flow Cytometry.

To evaluate the percentage of cells in the G₀/G₁, S, or G₂/M phase, flow cytometry profiles were determined on propidium iodide stained DNA from said cells. To this end, the cells were trypsinized, centrifuged, resuspended in 300 μl of PBS and cooled on ice. 700 μl of 95% ethanol were then added dropwise while stirring the cells gently on a vortex, and the cells were fixed in ice for 30 min. After centrifugation and washing with PBS, the cells were incubated in 500 μl of PBS containing 40 U/ml of RNase (“DNAse free”, Roche Diagnostics, Laval, Quebec, CA) and 50 μg/ml of propidium iodide (Sigma-Aldrich CA Ltd.) for 30 min at room temperature in the dark. DNA measurements were carried out on an Epics XL (Beckman Coulter, Mississauga, CA) at an excitation wavelength of 488 nm and an emission wavelength of 620 nm. The percentage of cells in the G1, S, and G₂/M phase was quantified with Multicycle AV software (Phoenix Flow System, San Diego, Calif.) (28).

12. Transfections of HeLa and T47-D Cells.

TReP-132 activation of the p21, p27 and p16 cyclin kinase inhibitor promoters was studied by transfection of HeLa and T47-D cells. HeLa cells were seeded in 24-well plates at an initial density of 15,000 cells per well and transfected the next day with ExGen 500 (MBI Fermentas, Flamborough, Ontario, Canada) at a ratio of 4 μl of ExGen 500 per 0.5 μg DNA. T47-D cells were seeded at a density of 100,000 cells per well and transfected the next day with FuGENE 6 reagent (Roche Molecular Biochemicals, Laval, Canada), at a ratio of 3 μl of FuGENE per 1 μg of DNA. After a 12-hour incubation at 37° C., the medium was replaced and the cells were incubated for another 8 hours, then lysed for 15 minutes at room temperature in a solution containing 0.8% Triton X-100, 25 mM glycylglycine pH 7.8, 15 mM MgSO4, and 4 mM EGTA. Luciferase activity was assayed on a Berthold Lumat LB9501 luminometer (Berthold Detection Systems GmbH, Pforzheim, Germany) using the Dual-Luciferase™ reporter assay system kit (Promega Corporation, Madison, Wis.) enabling the simultaneous assay of firefly and Renilla luciferase activity (encoded by plasmid pRL-null for normalization). For all transfections, 0.1 μg of reporter plasmid containing the promoter under study (p21^{WAF1/CIP1}, p27^{Kip1}, or p16^{CDKN2}) and 0.01 μg of pRL-null were used. The amounts of TReP-132, p300, and Sp1 expression vectors varied according to the experiment and are indicated in the “Results” section. Each experiment included a control transfection with pcDNA3-EGFP and flow cytometry analysis confirmed that at least 60% of cells were transfected.

13. RNase Protection Assays in HeLa Cells.

The radiolabelled TReP-132 probe was prepared as described earlier: the pcDNA3-TReP-132-HA vector was first linearized by digestion with the restriction enzyme Narl (New England Biolabs, Beverly, Mass.) prior to priming in vitro transcription from the T7 promoter in the presence of [α-³²P]UTP-radiolabelled ribonucleotide in the conditions specified in the MAXIscript kit (Ambion, Austin, Tex.). A 226 bp cRNA probe was generated, containing 174 nucleotide bases located at the 3′ end of TReP-132 and 52 bases originating from the polylinker and including the HA tag. The 18S riboprobe used for normalization was generated from the human pTRI-18S vector (Ambion, Austin, Tex.) and protected an 80 bp fragment. The ribonuclease protection experiments were carried out with the Ribonuclease II Protection kit (Ambion, Austin, Tex.) as directed by the manufacturer. Thirty micrograms of RNA from MCF-7 cells were hybridized with the two probes for 16 hours at 45° C. and the hybrids were then digested for 45 min at 37° C. in RNase A/RNase T1 solution diluted 1:75 in digestion buffer. The samples were separated on a polyacrylamide-6% urea gel.

All radiolabelled probes specific for the p130, Rb, p107, p53, p57, p27, p21, p19, p18, p14/p15, L32, and GAPDH genes were labelled with [α-³²P]UTP-ribonucleotide (PerkinElmer Life Sciences, Woodbridge, Ontario, CA) in the conditions specified in the “hCC-2 Muti-Probe Template Set” kit (Pharmingen, Mississauga, Ontario, CA). Ribonuclease protection experiments were carried out with the Ribonuclease III Protection kit (Ambion, Austin, Tex.). Ten micrograms of HeLa cell RNA were hybridized with the radiolabelled probe mixture at 56° C. and then digested for 45 min at 30° C. in RNase A/RNase T1 solution diluted 1:1000 in digestion buffer. The samples were separated on a polyacrylamide-6% urea gel.

14. Preparation of Protein Extracts, Analysis by Western Blot and Co-Immunoprecipitation.

Cells were washed twice with cold PBS (Phosphate Buffered Saline), then harvested either in cold RIPA buffer (1×PBS, 1% Nonidet P-40, 0.5% sodium deoxycholate, 0.1% SDS, phosphatase and protease inhibitors) for co-immunoprecipitation analysis, or in lysis buffer (50 mM Tris, pH 8.0, 0.1% IGEPAL (Sigma)) for kinase assays. The lysates were incubated for 30 min on ice and cell debris was eliminated by centrifugation. The extracts were then aliquoted and stored at −80° C. For western blot analysis, RIPA or lysate buffer was used. For immunoprecipitations, whole cell lysates (1 mg) in RIPA buffer were preincubated with 0.1 μg of mouse or rabbit IgG and 20 μl of protein A-Sepharose beads (Amersham Biosciences, Uppsala, Sweden) for 30 min at 4° C. The lysates (1 mg) were then centrifuged and immunoprecipitated for 1 hour at 4° C. with anti-CDK2 (sc-163) (Santa Cruz), anti-CDK6 (RB-017), anti-cyclin A (RB-010) or anti-cyclin D1 (MS-395) (Microm France, Francheville) antibody, then incubated overnight at 4° C. with protein A-Sepharose. The immunoprecipitated proteins were washed four times with lysis buffer, then resuspended in electrophoresis buffer. Protein samples (20-100 μg) were heated at 95° C. for 5 min, then separated by SDS-PAGE and transferred to nitrocellulose membranes previously saturated with powdered milk diluted in TBS buffer (10 mM Tris, pH 8.0, 150 mM NaCl) containing 0.2% Tween 20 (Sigma) and then incubated (2-4 hours at room temperature or overnight at 4° C.) with the antibodies anti-p21 (OP64) (Merck Eurolab, Fontenay-sous-Bois, France); anti-p27 (K25020, Transduction Laboratories, Lexington, Ky.); anti-pRb (554136, recognizing all phosphorylation states of pRb); anti-ppRb-Ser780 (sc-12901) specific for a pRb phosphorylated on Ser 780; anti-PR (MS-298-P) and anti-beta-actin (sc-7210) as control. The membranes were washed, then incubated with mouse or rabbit HRP-coupled secondary antibody (Jackson Immunoresearch Laboratories, West Grove, Pa.) for 45 min. The blots were then visualized with ECL reagent and quantified with SCANWISE and Perfect-IMAGE V-5.3 software (CLARA VISION, France).

15. Kinase Assays.

The kinase activities on histone H1 or Rb protein in the immunoprecipitates were measured as follows: the immunoprecipitates were first washed once with 1 ml of assay buffer (50 mM Tris-HCl, pH 7.4, 10 mM MgCl₂, 1 mM dithiothreitol (DTT)), then resuspended in 20 μl of assay buffer containing 1 mg/ml casein (Sigma) and 1 μg of histone H1 or Rb protein (769) (sc-4112) as substrate. After a 5-min preincubation at 30° C., the reaction was initiated by adding 1 μM ATP and 5 μCurie of [gamma-³²P] ATP, then continued at 30° C. for 1 hour.

The reaction was stopped by addition of 15 μl of electrophoresis buffer (3×). The samples were run on a 10% SDS-PAGE gel. The gel was dried and autoradiographed. Phosphorylated Rb and histones were quantified by densitometry.

16. In Vitro Binding Assays.

In order to evaluate the interaction of TReP-132, Sp1, ER and PR, the wt and mutant GST-TReP-132 fusion proteins were expressed, then immobilized on glutathione-Sepharose as described (Frangioni and Neel, 1993; Monte et al., 1998). The ³⁵S-[Sp1], ³⁵S-[ER] and ³⁵S-[PR-B] proteins were synthesized in vitro using a rabbit reticulocyte lysate and T7 RNA polymerase system as directed by the manufacturer (Promega Corporation, Madison, Wis.). Proteins which bound, then detached from the Sepharose after being heated to boiling in SDS buffer were then separated on a 10% SDS-PAGE gel. The gels were stained with Coomassie blue, dried and autoradiographed.

17. Chromatin Immunoprecipitation (ChIP).

HTO cells incubated in the presence of Dox or T47-D cells were grown to 70% confluence in petri dishes (150 mm diameter). For HTO cells, doxycycline was removed from half of the dishes 48 hours before lysis. T47-D cells were incubated with 30 nM progesterone or with ethanol containing 0.2% BSA-RPMI for 2.5 hours before lysis. Cell lysates were then sonicated on ice, 15 times for 15 seconds at 45 second intervals. A volume of lysate corresponding to 4×10⁶ cells was immunoprecipitated by using 4 μg of antibody (indicated earlier) and an anti-HA antibody as negative control. The same volume was set aside for later purification of genomic DNA. One-twentieth of the DNA extract was then amplified by PCR for 35 cycles (30 sec at 92° C., 30 sec at 55° C. and 30 sec at 72° C.) using the primers: for p21, 5′-ggcactcttgttcccccaggc-3′ and accatccccttcctcacctg-3′ (Sp1 response element on p21) or 5′-gcacactgacgcagcacacag-3′ and 5′-cagtttgagaagcagccacct-3′ (distal p21 element); for p27, 5′-aggccagccagagcaggtttgt-3′ and 5′-ggaggagatccattggttgcgg-3′ (Sp1 response element on p27) or 5′-gacttgcatctagtcctgactccgg-3′ and 5′-gcctacctcatctcatacgctccag-3′ (distal p27 element); and for beta-actin, 5′-aaactctccctcctcctcttcct-3′ and 5′-cgagccataaaaggcaactttcg-3′. An equivalent volume of unprecipitated genomic DNA was amplified as negative control. One-fifteenth (unprecipitated genomic DNA) or one-fifth (precipitated DNA) of the PCR products were then separated on a 2% agarose gel containing ethidium bromide.

18. FRET

In order to evaluate the effect of a ligand on the recruitment of TReP-132 by ER and PR, FRET (Fluorescence Resonance Energy Transfer) was carried out between TReP-132 and PR or ER.

GST-LBD ER and GST-LBD PR fusion proteins (“GST” for Gluthatione-S-Transferase and “LBD” for Ligand Binding Domain) were expressed and purified with the aid of GST-Trap columns (Amersham Pharmacia).

The two NR-box peptides (nuclear receptor binding domain) of TReP-132 (NH2-AVMDGAPDSALRQLLQKPMEPPAPA-COOH and NH2-FEAKGDVMVALEMLLLRKPVRLKCH-COOH) were synthesized and labelled with biotin. GST-LBD ER, GST-LBD PR and NR-box peptides were respectively detected with AlloPhycoCyanin (APC) coupled with anti-GST antibody and with R-phycoerythrin (RPE) coupled with streptavidin.

Increasing concentrations of compounds were incubated in binding buffer (0.1 M PBS, 2 mM CHAPS, 2 mM EDTA, 1 mM DTT, 0.1% BSA, pH 7.2) with 35 nM GST-LBD ER or GST-LBD PR, 26.3 nM APC-labelled anti-GST antibody, 1.25 nM RPE-strepavidin, 5-30 nM biotinylated NR-box peptide, at 4° C. for 4 hours in 384-well plates. The excitation wavelength for RPE was 495 nm and emission was measured at 635 nm (RPE) or 670 nm (APC). Fluorescence intensities were measured on a Genesis Freedom 200 Tecan. The fluoresence ratios (intensity at 670 nm/intensity at 635 nm) relative to the ligand were calculated.

### Results

1—Effect of TReP 132 on Cell Proliferation.

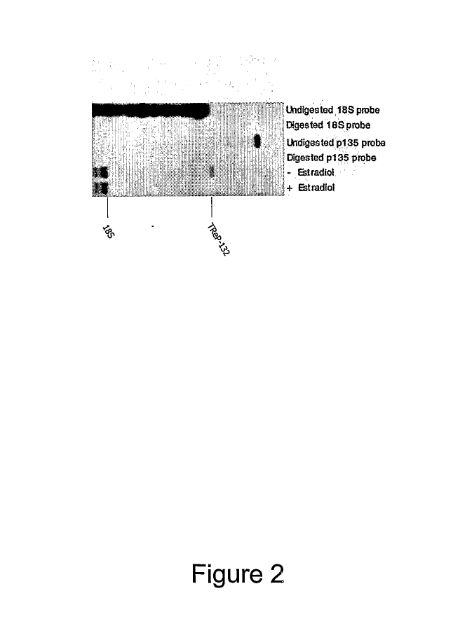

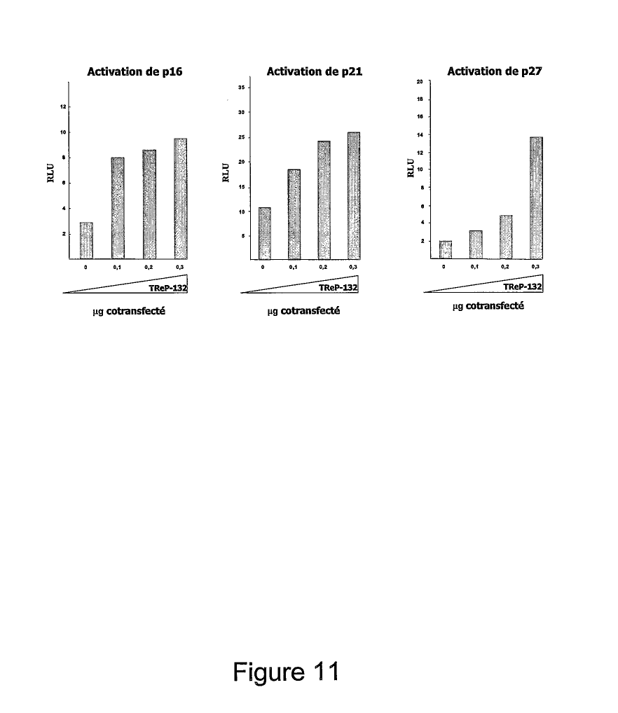

The effect of TReP-132 on cell proliferation was first demonstrated in HeLa cells by a colony formation assay. This experiment showed that HeLa cells transfected with a pcDNA3-TReP expression vector formed far fewer G418-resistant colonies than cells transfected with pcDNA3 alone (FIG. 11). 75 to 80% fewer colonies formed after transfection with the pcDNA3-TReP vector than after transfection with the control vector. Furthermore, when these cells were analyzed for expression of exogenous TReP-132, only two of 20 colonies were positive. When said two colonies were dispersed and reseeded, the cell proliferation rate had significantly decreased. The doubling time of cells expressing TReP-132 was 30 hours versus 18 hours for control cells transfected with the pcDNA3 vector alone. Thus, these data show that the increase in TReP-132 expression is correlated with the decrease in cell proliferation and therefore is probably responsible for the smaller number of viable colonies. To more clearly demonstrate that TReP-132 expression is closely correlated with cell proliferation, human breast cancer MCF-7 cells were treated for 6 days with 10 μM estradiol (E₂), a compound known to exert a proliferative effect on estrogen-dependent cells such as breast cancer cells via induction of the G₁→S transition. Following treatment of MCF-7 cells with estradiol, RNase protection assays revealed a marked decrease in TReP-132 expression (FIG. 2) and a concomitant increase in cell proliferation.

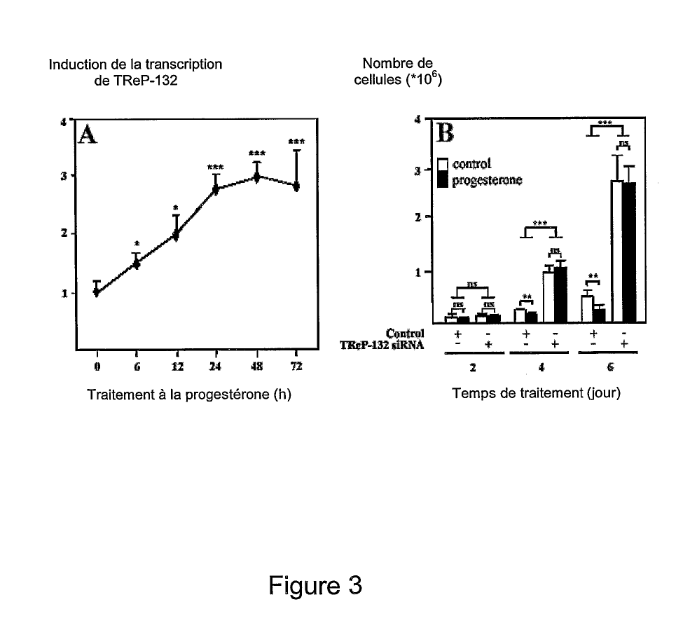

The effect of TReP-132 progesterone-dependent cell proliferation was demonstrated by measuring TReP-132 mRNA levels in T47-D cells treated or not with progesterone. TReP-132 has previously been shown to be a steroidogenic factor expressed in T47-D breast cancer cells (Musgrove et al., 1997) expressing high levels of PR (Progesterone Receptor) (Mockus et al., 1982). The results in FIG. 3A show that TReP-132 mRNA levels increased with exposure time to progesterone, reaching a peak (3-fold) at 48 hours of exposure. These data thus show that TReP-132 expression is increased by treating T47-D cells with progesterone.

The effect of TReP-132 on cell proliferation and response to progesterone was then demonstrated by the Small Interfering RNA (siRNA) method. FIG. 3B shows that progesterone inhibited cell proliferation after 4 to 6 days of treatment in transfected control cells, whereas TReP-132 knockdown led to an inhibition of cell proliferation, even in untreated cells. These data indicate that TReP-132 is a cell growth inhibitory factor and that TReP-132 acts as a mediator of the anti-proliferative effect of progesterone.

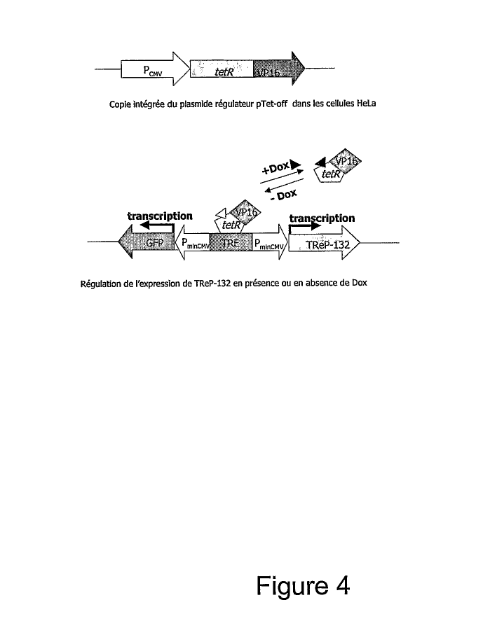

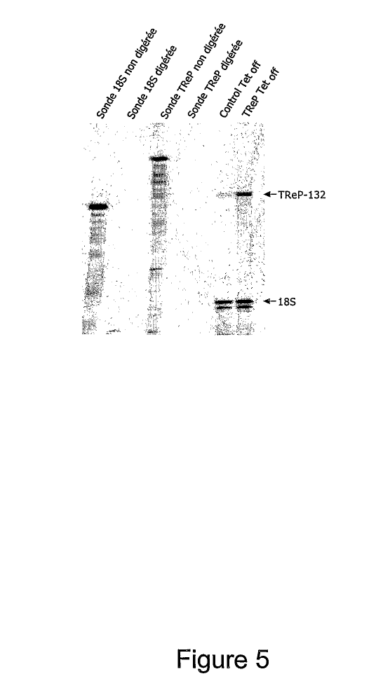

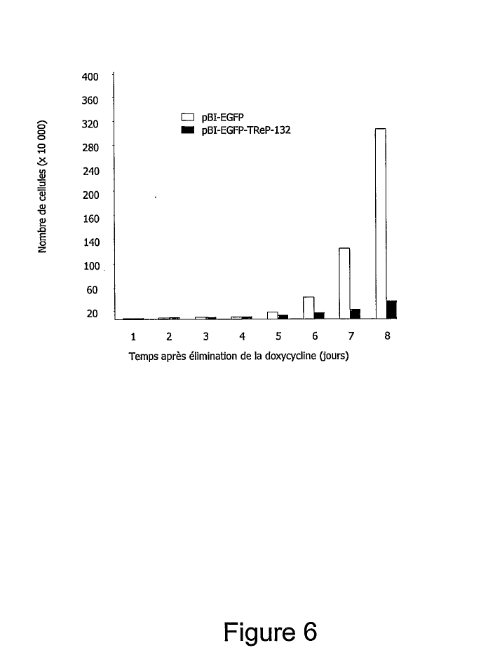

In order to study the mechanism of action of TReP-132 in cell growth control, clones with stable inducible expression of TReP-132 via the Tet-off system were produced in HeLa cells (FIG. 4). In this manner, cell lines co-expressing GFP and TReP-132-Flag (HTO-GFP/TReP) or only expressing GFP (HTO-GFP) were established. In this system, the cells were cultured in the presence of doxycycline so as to inhibit expression of TReP-132. The TReP-132-Flag protein was not detectable in cells cultured in the presence of Dox. On the other hand, when dox was removed from the medium, TReP-132-flag protein expression was detectable at 12 hours and increased up to 48 hours (FIG. 5). Thus said cell line constitutes a suitable model to study the effects of TReP-132 expression on cell proliferation. While HTO-GFP/TReP-132 cells cultured in the presence of Dox resumed a normal cell rate with a doubling time of about 16 hours, after 4 days of culture, removal of Dox considerably reduced cell proliferation to a doubling time of approximately 60 hours (FIG. 6). Since no differences in cell proliferation were observed when HTO-GFP cells and parent HTO cells were incubated with or without Dox, it can be concluded that overexpression of TReP-132 results in a strong inhibitory effect on HeLa cell proliferation.

2—Effect of TReP-132 on the Cell Cycle: Arrest in the G₁ Phase.

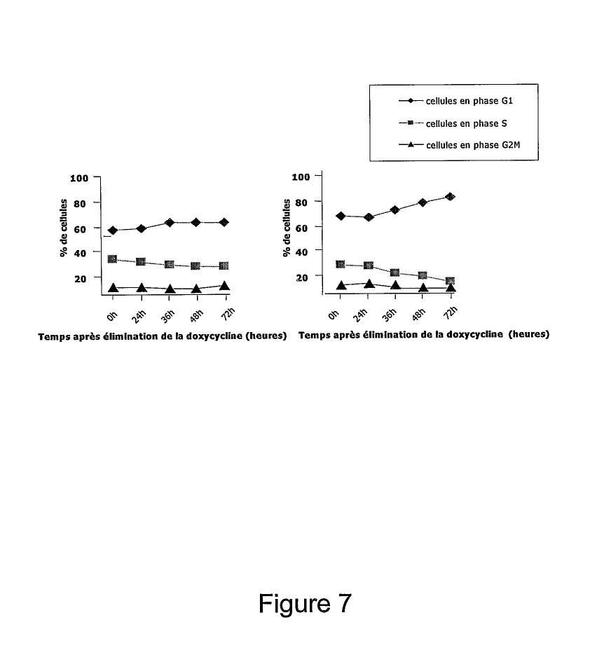

If TReP-132 is indeed involved in regulating cell cycle progression, it can be expected that the expression thereof will also be regulated during the different phases of the cell cycle. To address this question, flow cytometry analysis of propidium iodide-stained cellular DNA showed that induction of TReP-132 expression arrested cells in the G₁ phase (FIG. 7). In fact, starting from 48 hours after doxycycline removal, which corresponds to optimal induction of TReP-132 expression, an increase in the proportion of cells in the G₁ phase and a concomitant decrease in the proportion of cells in the S phase were observed.

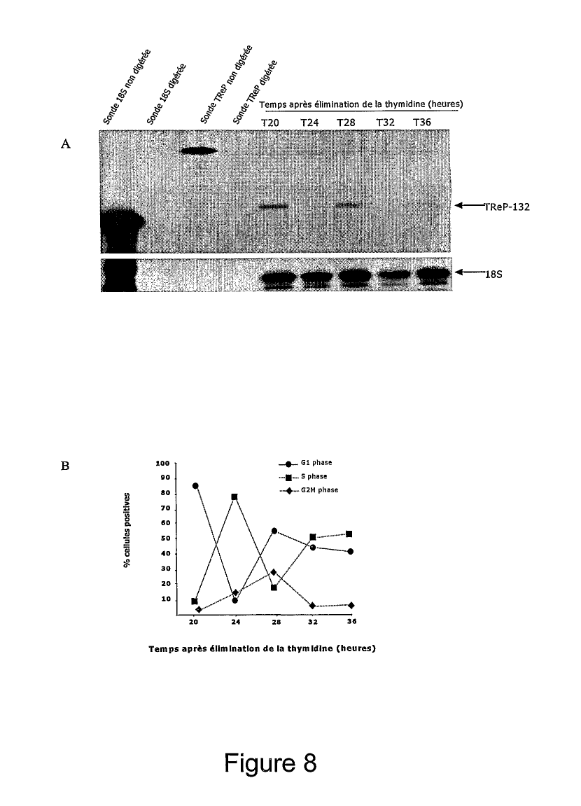

In addition, HeLa cells were synchronized at the end of the G₁ phase by a double thymidine block. Twenty hours after the second incubation with thymidine, which corresponds to time 0 in these experiments, the cells were harvested every 4 hours for cell cycle analysis by flow cytometry and for TReP-132 expression by RNase protection assays. TReP-132 expression was induced at times T20 and T28, when the majority of cells were in the G₁ phase (88 and 57% of cells, respectively) (FIG. 8). In contrast, TReP-132 expression was not detectable when 82% of cells were in S phase, at time T24. This experiment shows that TReP-132 expression is dependent on the G₁ phase of the cell cycle, which is in agreement with the ability of said protein to arrest cell cycle progression in this phase.

These data reveal an inverse correlation between TReP-132 expression and cell cycle progression.

3-TReP-132 Expression Leads to Inhibition of CDK Activities (Cyclin Dependent Kinases) and to a Decrease in Rb Phosphorylation (Retinoblastoma Protein).

Cell cycle progression is carried out by cyclin-dependent kinases (CDKs) which phosphorylate a number of key proteins involved in cell cycle control (Schafer, 1998). As CDKs are constitutively expressed during the cell cycle, the regulation of their activity, which occurs only at specific steps of the cell cycle, is ensured by post-transcriptional mechanisms. For instance, CDKs are activated only by interaction with specific cyclins, cyclins A, B, C, D1, or E, so named due to their cyclic expression during the cell cycle. Moreover, CDKs are inhibited by interaction with CDK inhibitors (CDKIs), which compete for binding to cyclins and prevent their resultant activation. CDKI expression is also regulated during the cell cycle, as well as by growth factors (Owa et al., 2001), and in particular those of the insulin family (Stewart et al., 1990). It has also been shown that the respective levels of CDKIs (and cyclins) are altered by irradiating cells, thereby confirming their involvement in processes associated with tumor development (Bernhard et al., 1995). The fact that TReP-132 was initially characterized as a transcription factor (Gizard et al., 2001) would suggest that it controls cell cycle progression by regulating the expression of proteins which are transiently induced during the cell cycle.

Progression through the cell cycle is controlled by a family of CDKs whose activity is regulated by phosphorylation, activated by binding to cyclins and inhibited by CDK inhibitors, which include the family of “CDK4 inhibitors” (“INK4”) (p16^{INK4A}, p15^{INK4B}, p18 and p19) and the family of “protein kinase inhibitors” (“PKI”) (p21^{WAF/CP-1}, p27^{KiP1} and p57^{Kip2}) (Sherr, 1994; Morgan, 1995; Sherr and Roberts, 1999). A key target of CDK action during the G₁ phase is the product of the retinoblastoma susceptibility gene (pRb) which induces G₁ arrest by sequestering transcription factors from the E2F-DP family. Phosphorylation of pRb and other members of this family such as p107 and p130, through active cyclin-CDK complexes, leads to release of E2F and DP and to the resultant transcriptional regulation of target genes required to enter the S phase.

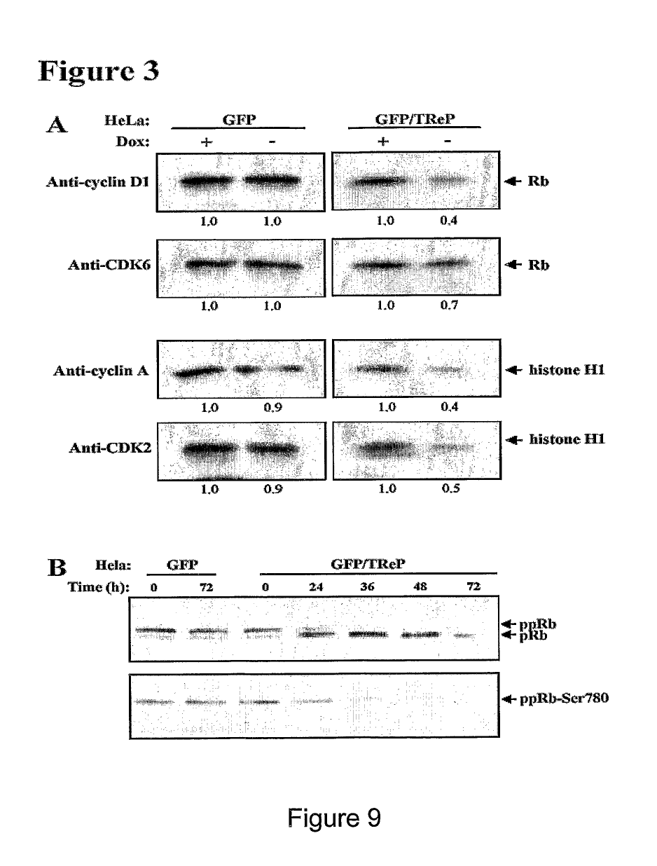

Phosphorylation of pRb and histone H1 by CDKs in the G₁ phase is a major event for progression of cells to the S phase (Peter and Herskowitz, 1994; Zarkoska and Mittnacht, 1997). To determine whether TReP-132 influences cyclin/CDK complex activities, the in vitro kinase activities of immunoprecipitated cyclin D1/CDK6 and cyclin A/CDK2 complexes were determined using pRb and histone H1 as substrate, respectively (Kitagawa et al., 1996; Koff et al., 1991). The results, presented in FIG. 9A, show that induction of TReP-132 expression in HTO-GFP/TReP-132 cells for 48 hours reduced the in vitro kinase activities of the cyclin/CDK complexes immunoprecipitated in the G₁ phase (cyclin D1/CDK6 and cyclin A/CDK2).

Analyses performed directly on total protein extracts from HTO cells, shown in FIG. 9B, indicate that while pRb was mainly present in an inactive, hyperphosphorylated state (ppRb) in uninduced HTO cells expressing GFP and TReP-132 under control of doxycycline, TReP-132 expression led to a significant increase in the amount of hypophosphorylated pRb (pRb) and a marked reduction in phosphorylated pRb (ppRb). Phosphorylated pRb levels decreased within 24 hours of Dox removal and were almost undetectable at 72 hours, whereas no change in pRb phosphorylation was observed in HTO cells expressing GFP alone. These data therefore show that hypophosphorylation of pRb reached its maximum at 36 hours, a key time point preceding the maximum accumulation of cells in the G₁ phase (observed at 48 hours), thereby demonstrating the role of TReP-132 in cell cycle arrest at the G₁ phase.

4-TReP-132 Upregulates the Expression of CDKIs (Cyclin Dependent Kinase Inhibitors).

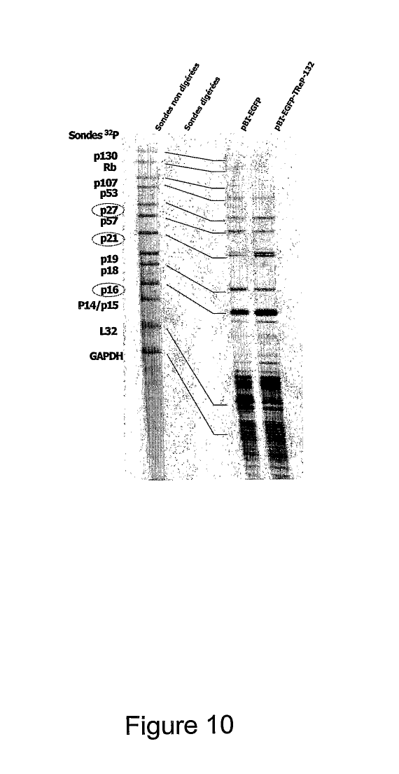

RNase protection assays were then carried out to determine whether induction of TReP-132 expression in HeLa cells was associated with changes in the transcription of genes coding for the following CDKIs: p57, p27, p21, p19, p18, p16 and p27/p15, and that of factors controlling progression of the G₁ phase: Rb, p130, p107 and p53. The results showed that induction of TReP-132 expression by removal of doxycycline from the medium led to induction of the p27^{KiP1}, p21^{CIP1/WAF1} and p16^{INK4} genes (FIG. 10). The fact that these three cyclin kinase inhibitors are involved in modulating G₁ progression correlates with the fact that TReP-132 expression is induced and that TReP-132 arrests the cell cycle at the G₁ phase.

The ability of TReP-132 to activate the promoters of these genes was then investigated. Increasing amounts of the pcDNA3-TReP-132 expression vector were cotransfected with each reporter construct controlled by the promoter regions of the p21, p16 and p27 genes. The p27 promoter showed dose-dependent activation by TReP-132, up to approximately 7-fold induction, after cotransfection with 0.3 μg of the TReP-132 expression vector (FIG. 11). The p21 and p16 promoters could also be activated to a lesser extent by TReP-132 with a maximum 2 to 3-fold induction relative to the control in the absence of TReP-132. These experiments clearly demonstrate the direct role of TReP-132 in the transcription of key cell cycle regulatory molecules.

5. Characterization of this Activation with TReP-132 Acting as Mediator on the p27 and p21 Promoters.

p21 and p27 are the CDKIs whose regulation has been most extensively studied, particularly in terms of their key role in the G₁→S transition of the cell cycle. In fact, they inhibit the cyclin/CDK2 and cyclin/CDK4 complexes whose activity is correlated with the onset of chromosome duplication.

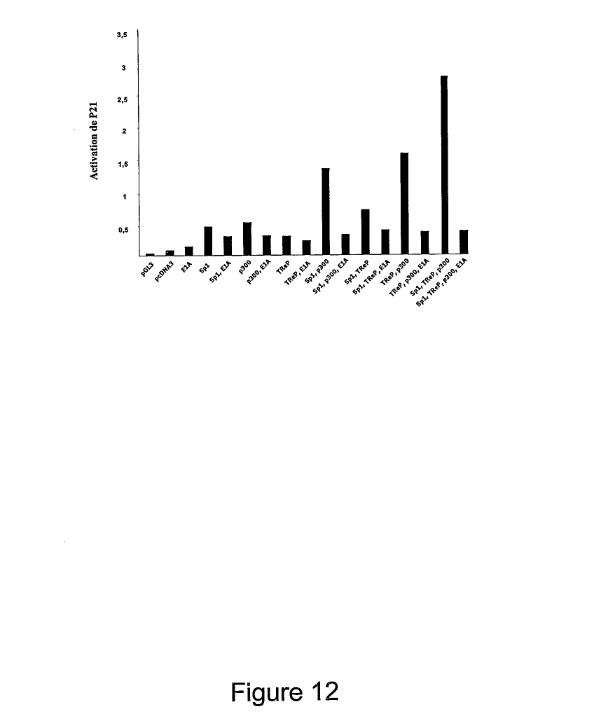

Transcription of p21 can be induced by progesterone. Owen et al. (1998) showed that said induction involves an interaction between the progesterone receptor (PR), CBP/p300 and the Sp1 transcription factor, at the level of Sp1 DNA binding sites in proximity to the TATA box. Since TReP-132 interacts with CBP/p300 to increase transcription of the P450scc gene (Gizard et al., 2001), it was determined whether TReP-132 induction of the p21 promoter similarly involved an interaction with CBP/300. To test this hypothesis, TReP-132, Sp1, or p300 expression vectors were cotransfected separately or simultaneously. As previously described by Owen et al. (1998), Sp1 and p300 can induce the p21 promoter and act additively when they are cotransfected simultaneously (FIG. 12). In the present invention, the inventors show that TReP-132 has an additive effect on the activation conferred by Sp1 or p300, and that maximum activation of the p21 promoter could be obtained when the three transcription factors were co-expressed. The specificity of the role of p300 in said activation was demonstrated by co-expression of E1A which is known to have an inhibitory effect on p300 (Frangioni and Neel, 1993; Monte et al., 1998). In the presence of E1A protein, the stimulatory effect was sharply reduced.

Previous studies have identified six proximal Sp1 binding sites in the −117/−51 region of the p21 promoter (Hong et al., 2002; Kardassis et al., 1999; Lu et al., 2000; Nakano et al., 1997; Pardali et al., 2000; Prowse et al., 1997; Santini et al., 2001; Somasundaram et al., 1997; Steger et al., 2002; Zhang et al., 2000) and two binding sites at positions −544 and −536 of the p27 promoter (Lee et al., 2003; Ryhanen et al., 2003; Williamson et al., 2002) which play a major role in the regulation of these genes.

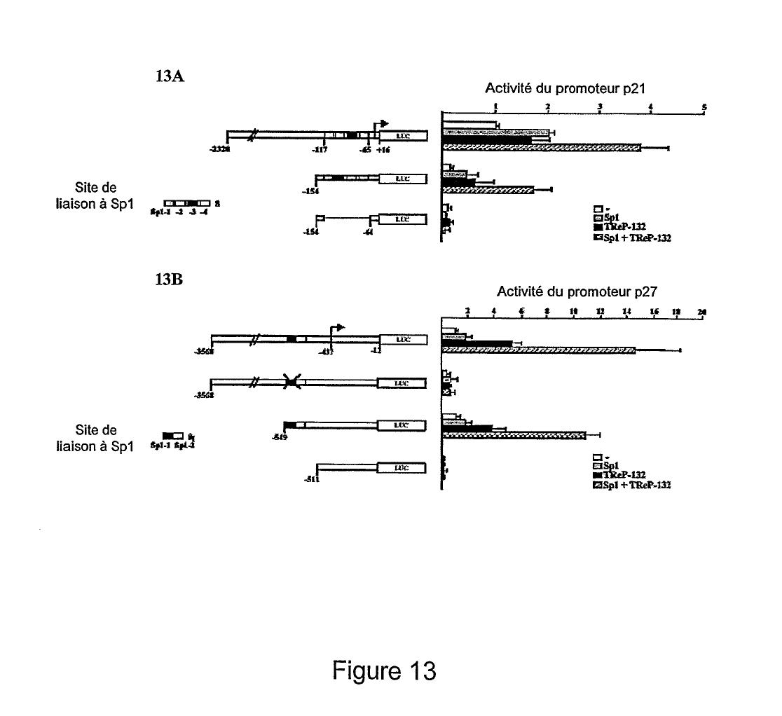

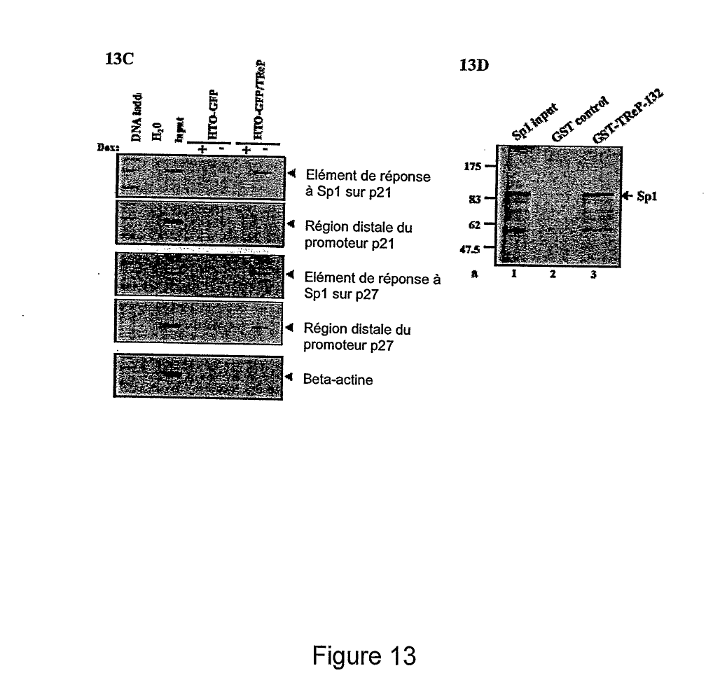

In order to study the mechanism of activation of the p21 and p27 genes by TReP-132, reporter constructs containing wt, mutant or deleted Sp1 response elements were cotransfected with Sp1 and/or TReP-132 expression vectors in HeLa cells. The results in FIGS. 13A and 13B show that, in a surprising manner, the activities of the −2320 p21 Luc and −3568 p27 Luc constructs were induced in the presence of Sp1 or TReP-132 alone, the highest activity being obtained when both factors were cotransfected. The results in FIG. 13B show that the shortest promoter construct (−154 p21 Luc) containing the Sp1 response element was activated by Sp1 and TReP-132 but the response to Sp1 and/or TReP-132 was abolished when the Sp1 response element was deleted. FIG. 13B shows that the −549 p27 Luc construct was still activated by Sp1 and/or TReP-132 whereas further deletions in the region comprising the Sp1 sites abolished activation by Sp1 or TReP-132, alone or together. Activation by Sp1 and TReP-132 was abolished by mutation of nucleotide −544 in the −3568 p27 Luc construct. These data therefore indicate that TReP-132 and Sp1 interact at proximal Sp1 binding sites of the p21 and p27 promoters.

Chromatin immunoprecipitation (ChIP) experiments were then performed to evaluate the interaction of TReP-132 and Sp1 with proximal Sp1 binding sites in the p21 and p27 promoters in cells. To this end, the experiments were carried out in HTO cells using an anti-Flag antibody to immunoprecipitate the Flag-TReP-132 fusion protein. The results are presented in FIG. 13C. The genomic DNA regions encompassing the Sp1 response elements of the p21 and p27 promoters were immunoprecipitated by the anti-Flag antibody in HTO cells expressing GFP and TReP-132 under doxycycline control in which TReP-132 expression was induced, but not in HTO-GFP cells. PCR amplification using primers covering a region approximately 1 kb upstream of the Sp1 response elements of the p21 and p27 promoters or primers specific for the beta-actin gene did not result in a significant signal, thus demonstrating the specificity of immunoprecipitation and PCR amplifications. These data show that TReP-132 is part of a complex binding to proximal Sp1 binding sites in the p21 and p27 promoters.

Gel shift assays using in vitro synthesized TReP-132 protein and double-stranded oligonucleotides encompassing the Sp1 sites on the p21 and p27 promoters were then carried out to determine whether TReP-132 directly binds to Sp1 elements. No DNA/protein complexes were detected, indicating that TReP-132 activates the promoter through interaction with DNA binding proteins other than Sp1. To test this hypothesis, pull-down assays were carried out with the GST or GST-TReP-132 fusion proteins and labelled Sp1 protein. The results, in FIG. 13D, show that a protein/protein itneraction was observed between Sp1 and the GST-TReP-132 fusion protein but no interaction was seen between Sp1 and the GST protein.

6—Interaction of TReP-132 with Progesterone and Estrogen Receptors.

TReP-132 mediation of the cell growth inhibitory effect of progesterone and the similar effects of TReP-132 and progesterone on the proliferation of cancer cells suggest that said two factors interact to regulate transcription of the p21 and p27 genes. Owen et al. (1998) has shown that the p21 gene is activated by the bound progesterone receptor (PR) through interaction with Sp1 at the third and fourth Sp1 sites (respectively Sp1-3 and Sp1-4).

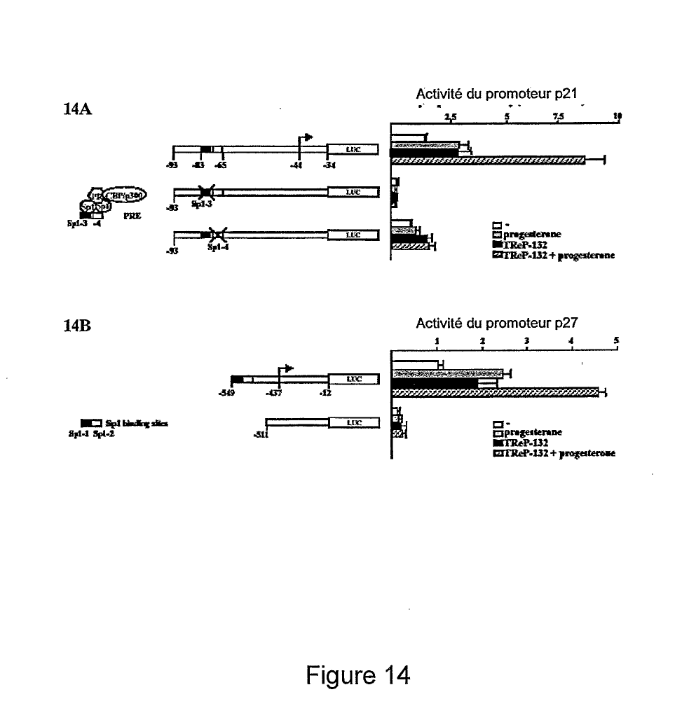

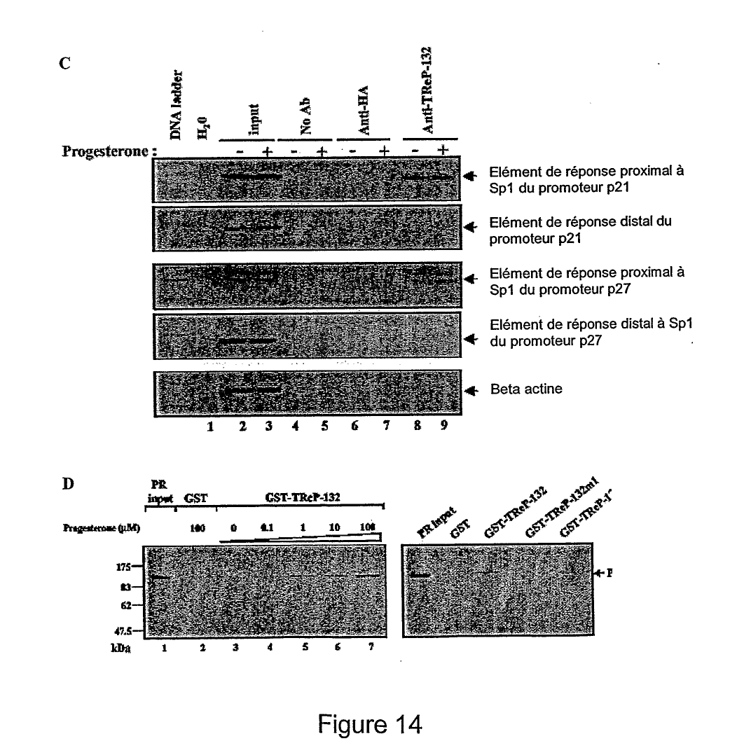

To test the interaction of TReP-132, PR and Sp1 at Sp1 binding sites on the two promoters, reporter constructs containing the two promoters were cotransfected with the TReP-132 expression vector in T47-D cells and the cells were treated or not with progesterone. The results, presented in FIGS. 14A and 14B, show that TReP-132 activated the two promoters, as did progesterone, whereas a higher level of activation was observed when cells were transfected with TReP-132 and also treated with progesterone. FIG. 14A shows that a mutation in the Sp1-3 site of the −93 p21 Luc construct completely abolished activation of the promoter by TReP-132 and progesterone, alone or in combination, while mutation of the Sp1-4 site reduced progesterone-dependent activation. Similarly, FIG. 14B shows that deletion of Sp1 binding sites in the p27 promoter led to a reduction of activation by TReP-132 and progesterone. These data indicate that TReP-132 might cooperate with PR and Sp1 to mediate the effects of progesterone on expression of the p21 and p27 genes. ChIP experiments were then carried out to test this hypothesis in cells, using DNA from T47-D cells treated or not with progesterone and anti-TReP-132 antibody. The results in FIG. 14C show that the proximal Sp1 response element of the p21 and p27 promoters was immunoprecipitated in a complex obtained by using anti-TReP-132 antibody. TReP-132 binding was much more pronounced with DNA from progesterone-treated cells. Negative controls were DNA extracts amplified with primers covering a region approximately 1 kb upstream of the Sp1 binding sites or primers specific for the beta-actin gene for which no product has been identified.

Pull-down assays then showed that TReP-132 and PR interact (FIG. 14D, column 3) and that said interaction is increased in a dose-dependent manner by progesterone (FIG. 14D, columns 3 to 7). TReP-132 contains two NR-boxes (Nuclear Receptor box) and LXXLL motifs at amino acids 181 (LRQLL) and 863 (LEMLL) (Gizard et al., 2001). Furthermore, it has been shown that the presence of the amino-terminal LXXLL motif is essential for interaction with SF-1 (Gizard et al., 2002b). Pull-down assays were then carried out with GST-TReP-132 proteins mutated at each LXXLL motif. FIG. 14D (right) shows that mutation of each motif led to a marked reduction in TReP-132 binding to PR, demonstrating the importance of LXXLL motifs in the in vitro interaction of TReP-132 with PR. Taken together, these data show that TReP-132 is a co-activator of PR in the regulatory complex formed with Sp1 at the proximal Sp1 binding site of the p21 and p27 promoters.

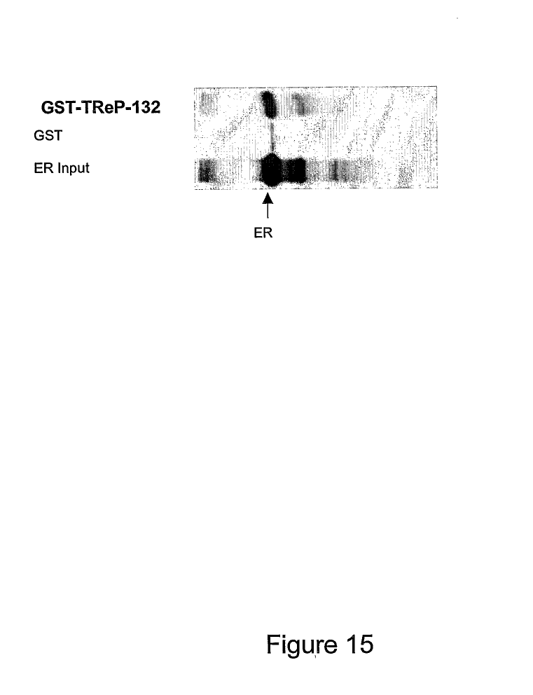

In the same manner, pull-down assays were carried out to test the interaction of TReP-132 and ER. The results in FIG. 15 show that TReP-132 and ER interact (line 3). From these data, together with the results showing a marked decrease in TReP-132 expression associated with an increase in cell proliferation in MCF-7 cells treated with estradiol, it can be concluded that the action of ER on cell proliferation takes place through a mechanism involving an interaction with TReP-132.

Additional FRET (Fluorescence Resonance Energy Transfer) studies also demonstrated the effect of a ligand on the recruitment of TReP-132 by ER and PR: the energy transfer between AlloPhycoCyanin (APC)-coupled anti-GST antibody to GST-PR or GST-ER fusion proteins, on the one hand, and streptavadin-coupled R-phycoerythrin (RPE) to the biotin/TReP-132 NR-box complex on the other hand, demonstrated that binding between TreP-132 and the ER and PR receptors is ligand-dependent.

### BIBLIOGRAPHY

Bernhard, E., A. Maity, et al. (1995). “Effects of ionizing radiation on cell cycle progression. A review.” Radiat Environ Biophys 34(2): 79-83.
Blais, A., D. Monte, et al. (2002). “Regulation of the Human Cyclin-dependent Kinase Inhibitor p18INK4c by the Transcription Factors E2F1 and Sp1.” J Biol Chem 277(35): 31679-31693.
Bloom, H. and W. Richardson (1957). “Histological grading and prognosis in breast cancer; a study of 1409 cases of which 359 have been followed for 15 years.” Br J Cancer 11(3): 359-77.
Cao, G., L. Liu, et al. (1991). “Modified method of mammalian cell synchronization improves yield and degree of synchronization.” Exp Cell Res 193(2): 405-10.
Contesso, G., H. Mouriesse, et al. (1987). “The importance of histologic grade in long-term prognosis of breast cancer: a study of 1,010 patients, uniformly treated at the Institut Gustave-Roussy.” J Clin Oncol 5(9): 1378-1386.
Duguay, Y., A. Lapointe, et al. (2003). “Cloning of murine TReP-132, a novel transcription factor expressed in brain regions involved in behavioral and psychiatric disorders.” Mol Psychiatry 8(1): 39-49.
Frangioni, J. and B. Neel (1993). “Solubilization and purification of enzymatically active glutathione S-transferase (pGEX) fusion proteins.” Anal Biochem 210(1): 179-87.
Gizard, F., M. El-Alfy, et al. (2002a). “Function of the transcriptional regulating protein of 132 kDa (TReP-132) on human P450scc gene expression.” Endocr Res 28(4): 559-74.
Gizard, F., B. Lavallee, et al. (2001). “A Novel Zinc Finger Protein TReP-132 Interacts with CBP/p300 to Regulate Human CYP11A1 Gene Expression.” J Biol Chem 276(36): 33881-33892.
Gizard, F., B. Lavallee, et al. (2002b). “The Transcriptional Regulating Protein of 132 kDa (TReP-132) Enhances P450scc Gene Transcription through Interaction with Steroidogenic Factor-1 in Human Adrenal Cells.” J Biol Chem 277(42): 39144-39155.
Gizard, F., E. Teissier, et al. (2004). “The transcriptional regulating protein of 132 kDa (TReP-132) differentially influences steroidogenic pathways in human adrenal NCI-H295 cells.” J Mol Endocrinol 32(2): 557-69.
Hong, C., H.-A. Kim, et al. (2002). “3,3′-Diindolylmethane (DIM) induces a G1 cell cycle arrest in human breast cancer cells that is accompanied by Sp1-mediated activation of p21WAF1/CIP1 expression.” Carcinogenesis 23(8): 1297-1305.
Ingle, J. (2002). “Sequencing of hormonal therapy in breast cancer.” Breast J 8(6): 332-7.
Inoue, T., J. Kamiyama, et al. (1999). “Sp1 and NF-Y Synergistically Mediate the Effect of Vitamin D3 in the p27Kip1 Gene Promoter That Lacks Vitamin D Response Elements.” J Biol Chem 274(45): 32309-32317.
Kardassis, D., P. Papakosta, et al. (1999). “c-Jun Transactivates the Promoter of the Human p21WAF1/Cip1 Gene by Acting as a Superactivator of the Ubiquitous Transcription Factor Sp1.” J Biol Chem 274(41): 29572-29581.
Kitagawa, M., H. Higashi, et al. (1996). “The consensus motif for phosphorylation by cyclin D1-Cdk4 is different from that for phosphorylation by cyclin A/E-Cdk2.” EMBO J 15(24): 7060-7069.
Koff, A., F. Cross, et al. (1991). “Human cyclin E, a new cyclin that interacts with two members of the CDC2 gene family.” Cell 66(6): 1217-28.
Lee, T., H. Chang, et al. (2003). “Involvement of PKA and Sp1 in the induction of p27(Kip1) by tamoxifen.” Biochem Pharmacol 66(3): 371-7.
Lu, S., G. Jenster, et al. (2000). “Androgen Induction of Cyclin-Dependent Kinase Inhibitor p21 Gene: Role of Androgen Receptor and Transcription Factor Sp1 Complex.” Mol Endocrinol 14(5): 753-760.
Mellon, C., F. Umar, et al. (1993). “Stuttering as a phenotype for behavioral genetics research.” Am J Med Genet 48(4): 179-83.
Miller, W. (1988). “Molecular biology of steroid hormone synthesis.” Endocr Rev 9(3): 295-318.

Mockus, M., B. Lessey, et al. (1982). “Estrogen-insensitive progesterone receptors in a human breast cancer cell line: characterization of receptors and of a ligand exchange assay.” Endocrinology 110(5): 1564-1571.
Mommers, E., A. Leonhart, et al. (2001). “Similarity in expression of cell cycle proteins between in situ and invasive ductal breast lesions of same differentiation grade.” J Pathol 194(3): 327-33.
Monte, D., F. DeWitte, et al. (1998). “Regulation of the Human P450scc Gene by Steroidogenic Factor 1 Is Mediated by CBP/p300.” J Biol Chem 273(8): 4585-4591.

Morgan, D. (1995). “Principles of CDK regulation.” Nature 374(6518): 131-4.
Musgrove, E. and R. Sutherland (1994). “Cell cycle control by steroid hormones.” Semin Cancer Biol 5(5): 381-9.
Musgrove, E. A., C. S. L. Lee, et al. (1997). “Antiprogestin Inhibition of Cell Cycle Progression in T-47D Breast Cancer Cells Is Accompanied by Induction of the Cyclin-Dependent Kinase Inhibitor p21.” Mol Endocrinol 11(1): 54-66.
Musgrove, E. A., A. Swarbrick, et al. (1998). “Mechanisms of Cyclin-Dependent Kinase Inactivation by Progestins.” Mol Cell Biol 18(4): 1812-1825.
Nakano, K., T. Mizuno, et al. (1997). “Butyrate Activates the WAF1/Cip1 Gene Promoter through Sp1 Sites in a p53-negative Human Colon Cancer Cell Line.” J Biol Chem 272(35): 22199-22206.
Owa, T., H. Yoshino, et al. (2001). “Cell cycle regulation in the G1 phase: a promising target for the development of new chemotherapeutic anticancer agents.” Curr Med Chem 8(12): 1487-503.
Owen, G. I., J. K. Richer, et al. (1998). “Progesterone Regulates Transcription of the p21WAF1 Cyclin-dependent Kinase Inhibitor Gene through Sp1 and CBP/p300.” J Biol Chem 273(17): 10696-10701.
Pardali, K., A. Kurisaki, et al. (2000). “Role of Smad Proteins and Transcription Factor Sp1 in p21Waf1/Cip1 Regulation by Transforming Growth Factor-beta” J Biol Chem 275(38): 29244-29256.
Peter, M. and 1. Herskowitz (1994). “Joining the complex: cyclin-dependent kinase inhibitory proteins and the cell cycle.” Cell 79(2): 181-4.
Peyrat, J., J. Bonneterre, et al. (1988). “Insulin-like growth factor 1 receptors in human breast cancer and their relation to estradiol and progesterone receptors.” Cancer Res 48(22): 6429-6433.
Prowse, D. M., L. Bolgan, et al. (1997). “Involvement of the Sp3 Transcription Factor in Induction of p21Cip1/WAF1 in Keratinocyte Differentiation.” J Biol Chem 272(2): 1308-1314.
Ryhanen, S., T. Jaaskelainen, et al. (2003). “Inhibition of MG-63 cell cycle progression by synthetic vitamin D3 analogs mediated by p27, Cdk2, cyclin E, and the retinoblastoma protein.” Biochem Pharmacol 66(3): 495-504.
Said, T. K., R. C. B. Moraes, et al. (2001). “Cyclin-dependent Kinase (cdk) Inhibitors/cdk4/cdk2 Complexes in Early Stages of Mouse Mammary Preneoplasia.” Cell Growth Differ 12(6): 285-295.
Santini, M. P., C. Talora, et al. (2001). “Cross talk among calcineurin, Sp1/Sp3, and NFAT in control of p21WAF1/CIP1 expression in keratinocyte differentiation.” PNAS 98(17): 9575-9580.
Schafer, K. A. (1998). “The cell cycle: a review.” Vet Pathol 35(6): 461-478.
Sherr, C. (1994). “G1 phase progression: cycling on cue.” Cell 79(4): 551-5.
Sherr, C. J. and J. M. Roberts (1999). “CDK inhibitors: positive and negative regulators of G1-phase progression.” Genes & Dev 13(12): 1501-1512.
Sitruk-Ware, R. and G. Plu-Bureau (1999). “Progestins and cancer.” Gynecol Endocrinol 13 Suppl 4: 3-9.
Somasundaram, K., H. Zhang, et al. (1997). “Arrest of the cell cycle by the tumour-suppressor BRCA1 requires the CDK-inhibitor p21WAF1/CiP1.” Nature 389(6647): 187-90.
Stahlberg, C., A. Pederson, et al. (2003). “Hormone replacement therapy and risk of breast cancer: the role of progestins.” Acta Obstet Gynecol Scand 82(7): 335-44.

Steger, G., C. Schnabel, et al. (2002). “The hinge region of the human papillomavirus type 8 E2 protein activates the human p21WAF1/CIP1 promoter via interaction with Sp1.” J Gen Virol 83(3): 503-510.
Stewart, A., M. Johnson, et al. (1990). “Role of insulin-like growth factors and the type I insulin-like growth factor receptor in the estrogen-stimulated proliferation of human breast cancer cells.” J Biol Chem 265(34): 21172-21178.
Stromstedt, M. and M. Waterman (1995). “Messenger RNAs encoding steroidogenic enzymes are expressed in rodent brain.” Brain Res Mol Brain Res 34(1): 75-88.
Thorpe, S. and A. Koenders (1986). “Standardization of steroid receptor assays in human breast cancer—III. Selection of reference material for intra- and inter-laboratory quality control.” Eur J Cancer Clin Oncol 22(8): 939-44.
Tobey, R. A., E. C. Anderson, et al. (1967). “The effect of thymidine on the duration of G1 in Chinese hamster cells.” J Cell Biol 35(1): 53-59.
Williamson, E., F. Dadmanesh, et al. (2002). “BRCA1 transactivates the cyclin-dependent kinase inhibitor p27(Kip1).” Oncoaene 21(20): 3199-206.
Zarkowska, T. and S. Mittnacht (1997). “Differential Phosphorylation of the Retinoblastoma Protein by G1/Cyclin-dependent Kinases.” J Biol Chem 272(19): 12738-12746.
Zhang, W., D. E. Geiman, et al. (2000). “The Gut-enriched Kruppel-like Factor (Kruppel-like Factor 4) Mediates the Transactivating Effect of p53 on the p21WAF1/Cip1 Promoter.” J Biol Chem 275(24): 18391-18398.

1-12. (canceled)

13. A method for the selection, identification, characterization or optimization of active compounds reducing cell proliferation, comprising determining the ability of a test compound to mimic, increase or promote the binding of the TReP-132 protein or of a complex comprising the TReP-132 protein to the receptor of one or more steroid hormones.

14. The method according to claim 13, comprising
a. contacting a test compound with:
i. the TReP-132 protein or a complex comprising the TReP-132 protein, and
ii. the receptor of one or more steroid hormones, and
b. measuring the binding of the TReP-132 protein or of the complex comprising the TReP-132 protein to said receptor.

15. The method according to claim 14, wherein the receptor is the progesterone receptor or the estrogen receptor.

16. A method for the selection, identification, characterization or optimization, in vitro or ex vivo, of active compounds reducing cell proliferation, comprising:
a. contacting, in the presence of progesterone or estrogens, a test compound with:
i. respectively, the progesterone receptor or the estrogen receptor,
ii. Sp1, and
iii. a nucleic acid comprising all or part of the promoter of a target gene of progesterone or estrogens, and
b. measuring the binding of Sp1 or of the complex comprising the progesterone or estrogen receptor and Sp1 to said promoter, or measuring the expression of said promoter, said measurement being an indication of the effect of the test compound on cell proliferation.

17. The method according to claim 16, wherein contact is carried out in the presence of the TReP-132 protein or a complex comprising the TReP-132 protein.

18. The method according to claim 16, wherein contact is carried out in a cell.

19. The method according to claim 16, wherein the target gene of progesterone or estrogens is selected in the group consisting of the p21, p16 and/or p27 genes.

20. A method for the treatment of a hormone-dependent cancer by administering to a subject in need of such treatment a compound selected from among the TReP-132 protein, an analog of said protein, a compound increasing or mimicking the activity of TReP-132 and a compound obtained with the aid of a method according to claim 13, in combination with one or more steroid hormones in view of a simultaneous, separate or sequential administration.

21. The method according to claim 20, wherein the hormone is progesterone or an estrogen.

22. The method according to claim 20, wherein hormone-dependent cancer is breast cancer or uterine cancer.

23. A pharmaceutical composition, comprising, in a pharmaceutically acceptable excipient, a compound selected from among the TReP-132 protein, an analog of said protein, a compound increasing or mimicking the activity of TReP-132 and a compound identified with the aid of a method according to claim 13, combined with one or more steroid hormones, in view of a simultaneous, separate or sequential administration for treating a hormone-dependent cancer.

24. The pharmaceutical composition according to claim 23, wherein the hormone is progesterone or an estrogen.
